SEQUENCE LISTING (22 sequences)

SEQ ID NO: 1 - DNA, 358 bp, Oryza sativa
ctgcaggcccagcaaacacgcgtcacacgtccccgtctttatctactcgcgcccatcctctacttatctactcggcctctcctcctccactactcctcgcctggtcctcccctctcctcgatcccctcttccccttcccgggcctctccgtggtggttggcgcgcggcggcaggcggaggcgggcggtggtgcgcggcggcgtgacataagcggcgggctgatgggagggcgggcggcgggtggaggggaggatggtggcgggcggatgggtgggcgacaacgggtgggcggcagcgggaggcggtgccctcccttctgccggaggggaggcgggaggcggcagcggcagcggcggat

SEQ ID NO: 2 - DNA, 2104 bp, Oryza sativa
actatgtttgtcggagtgtgtacatagatagggggccattcgatgggaccgctttctatgagttcgggggtgtcgcacgtgagggagcgctcgggttcccattgaggacggaaacaaatgaacaaacaataattatattagtttgagacatccggatgtgtaacatcctactcctattgtgtcatgtgttttctctcttgctagtgaatgatcttatatgtcagacttagagtcgtagttcatctcttttggatttccgaagaacttcactgtgtatggctatatgttctaataactctgtatgttcgaacctcctttctcttggtcatggctctcccattatatctatctagggacaccacatgatcagaatattagggagatgctaattaagtgcatagtgccggattatgtaaggaaaatcttgtcagcactgctcctctctccctttttgcgaggcttagcgtagaaccacgaaaaaaaaacttgtattaaagacaccaagataactagctgcaaacatcccagacaagccgatcatactcgaaatcaaccaccttctctatcacatcatattgaatttaaggtttatccaatcacattcaaagttactaactacagcaaccaggaaaatcctcgaaagaattagaagcatcaatgaagaagcattgaacatcacttctataatccctccaggaaaccatcagaagtaaaacattctctggcccccattggttgaatttttttctaataagccaaaacggcttattagagaataaaaataaatgtgtaggtaaaacttttatatatgtgttttttttaacttaaaagccaatactgaaaaaaactacgttgaaaatatctcagaatcaatctcaaaattaagtttgaaaattcaaaatttggcttattctttggcttattgggccatctgatgggagcctctatatttggtagcacataactaaataaataaaagggttcatattcctaattgtcgaaagtctctcaaagtgacataacacactagtatttacactacggacaaccctcctagtttagactttagagttacatgttgcccagtttcgaggtaaagtagactagctcaaatgtgtctcaaacagtcgttcccggaacataagtcacaagtgttcccacgtgtaggcatgtttcacgcttagatcgatcgagtttcgtttccatctgtacgtacgttgtaccaccccaacccgtcgatcgatatgatcgatcgtcccctataaattggacatcctggacatgctctttcttcagacatctccacccttcctatcttgatatataacttcgacacactgacaacttaatttgtcagaatgctcacggccacgcagactctggcgccggcagtgctctcccggagccatggcgccccttcttccttctccagccagccgcgccgcaccgccgccgccgcctcgagagtaagctgcacccgcgtcggcgccttgtcggaggtcgtcaatggcgaactcgtcgtcggcgaccaagaacagacgaccgacgacctccttacgcggcacaagaatgtcgtcgccgactacacgctgagcgccacggtgacggtgagcttgaagcaggacgattccactccccagaaggtggcggacatggttaatcgagactggcttttccttgatttcttcagctcgcatataggtgatgacaagctccatagctactctatcgatcgtaccggccgcctctttcgcgccatatataatcttgaacatgggcactgattcccattcgaaaaaatatcttaatttgagaggatgctttctactggtagctagcattctactactagaattatcgtattaaaagtgatcgcagccgttcgatttactagctagctcgaatgatagcatatattgtaagtgtaaattttgatacattttgatatataacttcgacattaggaaaaaaactacattaaaaattttggtacctaaaaaactataaaatttctactattatcagtaaaaatattttttgcttaccattagtagattagattgtatataagatatactacctctgttttttaatagatgacgcc

SEQ ID NO: 3 - DNA, 1516 bp, Oryza sativa
gtcatctattaaaaaacggagagagtatatgattcatcggaattaaaaaatagacggtataacacattgtaaaacacctattttcgtcactctacagaggggatgcacacggagcctcagctcgccaggtactcgcacatggatggcaaaggctccttcatatacgaggccagcttcagcatcccgtcctcgttggacgccgtcggcgccgtgcaggtcgtgaaccgctacagcagcgaggtgtacatctcggacatcgacgtccacctctgcggcggccgccatcagtggaccgacatcactttccactgcaactcttggatcgactacaaccccaacgaccagcgcttcttcttccctctcaaggtcaggggttcacgttattgccgataccgatattgttttattcgtgaggatggtaaaattagctatcggccaaaaattccttttttttttgaattagctcatatgtaaaataaaattaaaacctgcaaaccatcgaaaagggtggaggattatatttgcttaattagggcagtcccaacccataacactagacatggtttatataacactagacatcatcaagaaactagtactacacactactcttccaatgcaaataccactattccatacttcagttaaatgctacttatctcacatgatgtcttagatgttgtgtagaaaccatgtctcatgcaagacatggtttccttctctttctttatttattcacttgtcacattatatttttgtcctaggtggcaacttatttaatgctatggacaccattctagttattgggttgggaatgcccttaagggtgtgtttgattgggtggctgaagctgcatgggagttggggataaggcagccatctctcactagattggatgagtggatgaggagaacaccatgtgggtgatgttgctgattggctgtgataggtttaacttgatgttaacaatctcaaactgatttttttttctaaactgattatcctatttataatccgattatatgattatatttatattatagttaatcttcaaaacaaaatttcacatggttatacgaatgggtgccactgacttattgcgtccatgctatatcctccagcctatggatatatcaaacaaaacatagatgggttatcccatccccatccacttatgaccgtgaactaaacgcgccctaatctccatttccgctcgatcgatcgttgttcagctgcaaatcattcacactgcttgttatttgcttgcatgcacgatattgcagtcgtacctcccgtctcagacgcccaggggcgtgaagaatctgcgcaaggaagagctcagggccatccgcggcgatggccgcggcgagcgcaaggagtgggagcgcatctacgactacgacgtctacaacgacctcggcgaccccgacaatgacccggccactcgtcggccggtgctcggcggccgcgggcgcccctacccgcgccgctgccgcacgggccgccgccgctgcag

SEQ ID NO: 4 - DNA, 158 bp, Oryza sativa
ctatcttgatatataacttcgacacactgacaacttaatttgtcagaatgctcacggccacgcagactctggcgccggcagtgctctcccggagccatggcgccccttcttccttctccagccagccgcgccgcaccgccgccgccgcctcgagagta

SEQ ID NO: 5 - DNA, 3018 bp, Oryza sativa
ctatcttgatatataacttcgacacactgacaacttaatttgtcagaatgctcacggccacgcagactctggcgccggcagtgctctcccggagccatggcgccccttcttccttctccagccagccgcgccgcaccgccgccgccgcctcgagagtaagctgcacccgcgtcggcgccttgtcggaggtcgtcaatggcgaactcgtcgtcggcgaccaagaacagacgaccgacgacctccttacgcggcacaagaatgtcgtcgccgactacacgctgagcgccacggtgacggtgagcttgaagcaggacgattccactccccagaaggtggcggacatggttaatcgagactggcttttccttgatttcttcagctcgcatatagaggggatgcacacggagcctcagctcgccaggtactcgcacatggatggcaaaggctccttcatatacgaggccagcttcagcatcccgtcctcgttggacgccgtcggcgccgtgcaggtcgtgaaccgctacagcagcgaggtgtacatctcggacatcgacgtccacctctgcggcggccgccatcagtggaccgacatcactttccactgcaactcttggatcgactacaaccccaacgaccagcgcttcttcttccctctcaagtcgtacctcccgtctcagacgcccaggggcgtgaagaatctgcgcaaggaagagctcagggccatccgcggcgatggccgcggcgagcgcaaggagtgggagcgcatctacgactacgacgtctacaacgacctcggcgaccccgacaatgacccggccactcgtcggccggtgctcggcggccgcgggcgcccctacccgcgccgctgccgcacgggccgccgccgctgcaggacagacccgtcgtcggagtcgccgccggccaaggacggcgccgggatctacgtgccacgggacgaggcgttcacggagcggaaggccggcgcgttcgccaccaagaaggcgctgtcggcgctgtcggcgttcaccacggcgcagagggtgtccggcgaccggcggcggggcttcccgtcgctggcggccatcgacgcgctgtacgaggacgggtacaagaaccggccgtcgtcgtcgcagcaggaggcggacaacctcgaaggctacttcagggaggtgctccagaagcaggtgaagctgctgctcaagggcgagaaggaggagttcaaggaggagctacgcaaagtgttcaaattccaaacgcccgagattcacgacaaggacaagcttgcatggttcagagacgaggagttcgcgcggcaaacgctggcagggatgaaccctctcagcatccaacttgtcagggacacggacttccctatattcagcaagctggacgaggaaacctacggcccaggggactccctcatcaccaaagagctgattgaagagcagattaatggggtcatgacagcagaggaggccgtggagaagaagaagctgttcatgctggactaccacgacgtgctcctgccgttcgtgcacgcggtgcgcgagctggacgacaccacgctgtacgcctcgcggacgctcttcttcctgacggaggacggcacgctgaggccgatcgccatcgagctgacgaggcccaagtcccccaacacgccgcagtggcgccaggtcttcacgccgggctccagcgtcgcggcgtcctggctgtggcagctcgccaaaacgcacgtcctcgcccacgacaccggctaccaccagctcgtcagccactggctgaggacgcactgctgcgtggagccgtacgtgatcgcggcgaaccggcggctgagccagatgcaccccatctaccgactgctgcacccgcacttccgcttcaccatggagatcaacgcccaagcgcgcgggatgctcatcaacgccaatggaatcatcgagagcgccttcgcgccggggaagctctgcatggagctcagctcggcggtttacgacaagttttggaggttcgacatggaggctctgcccgccgatctcatccggaggggcatggcgatcgaatgcgaggatggcaagctggagctgacgatagaggactacccgtacgccaacgacggcctgctcatctgggactccatcaaggagtgggtgtcggattatgtgaaccattactaccagttggcttcagacatccacatggacaaggagctccagggttggtggaacgaggtgcgaaccaagggccacccggacaaggaggaagggtggccagagctgaactgccacgggagcctcgtcgaggttctgaccaccatcatctgggtcgcgtcggggcaccatgcggcggtgaactttggccagtacccctacgccggctacttccccaatcgccccaccatcgcccggcggaacatgccgacggaggggcaggcgtgcagtcacgacggcatgcagccaacgttcgttgaggatcccgtcagggtgctactagacacgttcccatcgcagtaccagaccaccctcgtcctgccggtgctcaacctgctatcgtcacactcgcccggcgaggagtacatgggcacgcatgcggagtcagcgtggatggcggacagggaggtcagggcggcgttcgggaggttcaacgagaggatgatgagcatcgcggagatgatcgactgccggaacaaggatccggagcgaaagaaccggcagggccccggcgtggtgccgtacgtgctgctcaagccgtcctacggtgaccctaaggacatgacgtccgtgatggagatgggtatccccaacagcatctcaatttgagttgtgccaatgagcttgcatctgtttggcgtgctcatcgtgacattatgtatgaaataaaatggattaaaaatccggcctcgtcaaggaatggctaacacagcgagcctgcatctgtttggagtgctcatcgtgcgattatgaaataaaatgacctggcatctgtttgccactgttttcttgtaaaaaaaaaaaaaaaaaa

SEQ ID NO: 6 - protein, 37 aa, Oryza sativa
MLTATQTLAPAVLSRSHGAPSSFSSQPRRTAAAASRV

SEQ ID NO: 7 - protein, 922 aa, Oryza sativa
MLTATQTLAPAVLSRSHGAPSSFSSQPRRTAAAASRVSCTRVGALSEVVNGELVVGDQEQTTDDLLTRHKNVVADYTLSATVTVSLKQDDSTPQKVADMVNRDWLFLDFFSSHIEGMHTEPQLARYSHMDGKGSFIYEASFSIPSSLDAVGAVQVVNRYSSEVYISDIDVHLCGGRHQWTDITFHCNSWIDYNPNDQRFFFPLKSYLPSQTPRGVKNLRKEELRAIRGDGRGERKEWERIYDYDVYNDLGDPDNDPATRRPVLGGRGRPYPRRCRTGRRRCRTDPSSESPPAKDGAGIYVPRDEAFTERKAGAFATKKALSALSAFTTAQRVSGDRRRGFPSLAAIDALYEDGYKNRPSSSQQEADNLEGYFREVLQKQVKLLLKGEKEEFKEELRKVFKFQTPEIHDKDKLAWFRDEEFARQTLAGMNPLSIQLVRDTDFPIFSKLDEETYGPGDSLITKELIEEQINGVMTAEEAVEKKKLFMLDYHDVLLPFVHAVRELDDTTLYASRTLFFLTEDGTLRPIAIELTRPKSPNTPQWRQVFTPGSSVAASWLWQLAKTHVLAHDTGYHQLVSHWLRTHCCVEPYVIAANRRLSQMHPIYRLLHPHFRFTMEINAQARGMLINANGIIESAFAPGKLCMELSSAVYDKFWRFDMEALPADLIRRGMAIECEDGKLELTIEDYPYANDGLLIWDSIKEWVSDYVNHYYQLASDIHMDKELQGWWNEVRTKGHPDKEEGWPELNCHGSLVEVLTTIIWVASGHHAAVNFGQYPYAGYFPNRPTIARRNMPTEGQACSHDGMQPTFVEDPVRVLLDTFPSQYQTTLVLPVLNLLSSHSPGEEYMGTHAESAWMADREVRAAFGRFNERMMSIAEMIDCRNKDPERKNRQGPGVVPYVLLKPSYGDPKDMTSVMEMGIPNSISI

SEQ ID NO: 8 - DNA, 17 bp, Artificial Sequence
  misc_feature (1)..(17): n=a,t, g or c
caygcngtnaanttygg

SEQ ID NO: 9 - DNA, 21 bp, Artificial Sequence
  Description of Artificial Sequence oligonucleotide
aatgctttttttttttttttv

SEQ ID NO: 10 - DNA, 20 bp, Artificial Sequence
  misc_feature (1)..(20): oligonucleotide
gtcagcatgctcacggccac

SEQ ID NO: 11 - DNA, 20 bp, Artificial Sequence
  misc_feature (1)..(20): oligonucleotide
cattgacgacctccgacaag

SEQ ID NO: 12 - DNA, 37 bp, Artificial Sequence
  misc_feature (1)..(37): oligonucleotide
caagaaagctgggttgacaaattaagttgtcagtgtg

SEQ ID NO: 13 - DNA, 37 bp, Artificial Sequence
  misc_feature (1)..(37): oligonucleotide
caaaaaagcaggcttgtaacatcctactcctattgtg

SEQ ID NO: 14 - DNA, 32 bp, Artificial Sequence
  misc_feature (1)..(32): oligonucleotide
caaaaaagcaggctccccgtctttatctactc

SEQ ID NO: 15 - DNA, 29 bp, Artificial Sequence
  misc_feature (1)..(29): oligonucleotide
ggggacaagtttgtacaaaaaagcaggct

SEQ ID NO: 16 - DNA, 29 bp, Artificial Sequence
  misc_feature (1)..(29): oligonucleotide
ggggaccactttgtacaagaaagctgggt

SEQ ID NO: 17 - DNA, 4569 bp, Oryza sativa
ctgcaggcccagcaaacacgcgtcacacgtccccgtctttatctactcgcgcccatcctctacttatctactcggcctctcctcctccactactcctcgcctggtcctcccctctcctcgatcccctcttccccttcccgggcctctccgtggtggttggcgcgcggcggcaggcggaggcgggcggtggtgcgcggcggcgtgacataagcggcgggctgatgggagggcgggcggcgggtggaggggaggatggtggcgggcggatgggtgggcgacaacgggtgggcggcagcgggaggcggtgccctcccttctgccggaggggaggcgggaggcggcagcggcagcggcggatttttttgttcttttttttttagtatttgtgattcattggatctgagatgtataatttgtgatgtatttttttttagaatttgtgatgtgaatctatgatttgtgatgttactttgatttggggatatgtagggagcaactcgatttggaaaaatcaaaatgcaagcagcaaagaaacaatgacaaaaaaagaagaaaaatggtgaccaaccgaaactgccgcaaccatcttttaatcccggttggtaacaccaaccagaactgaagatggacatctttaatttagtctcggattcacggtcctggtttacaacccgggactaaagggggttgcgaaccaggactaaagaagggttttccagcagttacagaaattaaaagtatatagtatttgctatatttactattttgccactatgtttgtcggagtgtgtacatagatagggggccattcgatgggaccgctttctatgagttcgggggtgtcgcacgtgagggagcgctcgggttcccattgaggacggaaacaaatgaacaaacaataattatattagtttgagacatccggatgtgtaacatcctactcctattgtgtcatgtgttttctctcttgctagtgaatgatcttatatgtcagacttagagtcgtagttcatctcttttggatttccgaagaacttcactgtgtatggctatatgttctaataactctgtatgttcgaacctcctttctcttggtcatggctctcccattatatctatctagggacaccacatgatcagaatattagggagatgctaattaagtgcatagtgccggattatgtaaggaaaatcttgtcagcactgctcctctctccctttttgcgaggcttagcgtagaaccacgaaaaaaaaacttgtattaaagacaccaagataactagctgcaaacatcccagacaagccgatcatactcgaaatcaaccaccttctctatcacatcatattgaatttaaggtttatccaatcacattcaaagttactaactacagcaaccaggaaaatcctcgaaagaattagaagcatcaatgaagaagcattgaacatcacttctataatccctccaggaaaccatcagaagtaaaacattctctggcccccattggttgaatttttttctaataagccaaaacggcttattagagaataaaaataaatgtgtaggtaaaacttttatatatgtgttttttttaacttaaaagccaatactgaaaaaaactacgttgaaaatatctcagaatcaatctcaaaattaagtttgaaaattcaaaatttggcttattctttggcttattgggccatctgatgggagcctctatatttggtagcacataactaaataaataaaagggttcatattcctaattgtcgaaagtctctcaaagtgacataacacactagtatttacactacggacaaccctcctagtttagactttagagttacatgttgcccagtttcgaggtaaagtagactagctcaaatgtgtctcaaacagtcgttcccggaacataagtcacaagtgttcccacgtgtaggcatgtttcacgcttagatcgatcgagtttcgtttccatctgtacgtacgttgtaccaccccaacccgtcgatcgatatgatcgatcgtcccctataaattggacatcctggacatgctctttcttcagacatctccacccttcctatcttgatatataacttcgacacactgacaacttaatttgtcagaatgctcacggccacgcagactctggcgccggcagtgctctcccggagccatggcgccccttcttccttctccagccagccgcgccgcaccgccgccgccgcctcgagagtaagctgcacccgcgtcggcgccttgtcggaggtcgtcaatggcgaactcgtcgtcggcgaccaagaacagacgaccgacgacctccttacgcggcacaagaatgtcgtcgccgactacacgctgagcgccacggtgacggtgagcttgaagcaggacgattccactccccagaaggtggcggacatggttaatcgagactggcttttccttgatttcttcagctcgcatataggtgatgacaagctccatagctactctatcgatcgtaccggccgcctctttcgcgccatatataatcttgaacatgggcactgattcccattcgaaaaaatatcttaatttgagaggatgctttctactggtagctagcattctactactagaattatcgtattaaaagtgatcgcagccgttcgatttactagctagctcgaatgatagcatatattgtaagtgtaaattttgatacattttgatatataacttcgacattaggaaaaaaactacattaaaaattttggtacctaaaaaactataaaatttctactattatcagtaaaaatattttttgcttaccattagtagattagattgtatataagatatactacctctgttttttaatagatgacgccgttgactttttctcacatgtttgaccattcgtcttattaaaaaattatataattataatttagtttgttatgaattgttttatcactcatagtactttaagtgtgatttatatcttatacatttacataaaatttttgaataagacgaatggtcaaacatgggagaaaaagtcaacggcgtcatctattaaaaaacggagagagtatatgattcatcggaattaaaaaatagacggtataacacattgtaaaacacctattttcgtcactctacagaggggatgcacacggagcctcagctcgccaggtactcgcacatggatggcaaaggctccttcatatacgaggccagcttcagcatcccgtcctcgttggacgccgtcggcgccgtgcaggtcgtgaaccgctacagcagcgaggtgtacatctcggacatcgacgtccacctctgcggcggccgccatcagtggaccgacatcactttccactgcaactcttggatcgactacaaccccaacgaccagcgcttcttcttccctctcaaggtcaggggttcacgttattgccgataccgatattgttttattcgtgaggatggtaaaattagctatcggccaaaaattccttttttttttgaattagctcatatgtaaaataaaattaaaacctgcaaaccatcgaaaagggtggaggattatatttgcttaattagggcagtcccaacccataacactagacatggtttatataacactagacatcatcaagaaactagtactacacactactcttccaatgcaaataccactattccatacttcagttaaatgctacttatctcacatgatgtcttagatgttgtgtagaaaccatgtctcatgcaagacatggtttccttctctttctttatttattcacttgtcacattatatttttgtcctaggtggcaacttatttaatgctatggacaccattctagttattgggttgggaatgcccttaagggtgtgtttgattgggtggctgaagctgcatgggagttggggataaggcagccatctctcactagattggatgagtggatgaggagaacaccatgtgggtgatgttgctgattggctgtgataggtttaacttgatgttaacaatctcaaactgatttttttttctaaactgattatcctatttataatccgattatatgattatatttatattatagttaatcttcaaaacaaaatttcacatggttatacgaatgggtgccactgacttattgcgtccatgctatatcctccagcctatggatatatcaaacaaaacatagatgggttatcccatccccatccacttatgaccgtgaactaaacgcgccctaatctccatttccgctcgatcgatcgttgttcagctgcaaatcattcacactgcttgttatttgcttgcatgcacgatattgcagtcgtacctcccgtctcagacgcccaggggcgtgaagaatctgcgcaaggaagagctcagggccatccgcggcgatggccgcggcgagcgcaaggagtgggagcgcatctacgactacgacgtctacaacgacctcggcgaccccgacaatgacccggccactcgtcggccggtgctcggcggccgcgggcgcccctacccgcgccgctgccgcacgggccgccgccgctgcag

SEQ ID NO: 18 - DNA, 1198 bp, Oryza sativa
tgtaacatcctactcctattgtgtcatgtgttttctctcttgctagtgaatgatcttatatgtcagacttagagtcgtagttcatctcttttggatttccgaagaacttcactgtgtatggctatatgttctaataactctgtatgttcgaacctcctttctcttggtcatggctctcccattatatctatctagggacaccacatgatcagaatattagggagatgctaattaagtgcatagtgccggattatgtaaggaaaatcttgtcagcactgctcctctctccctttttgcgaggcttagcgtagaaccacgaaaaaaaaacttgtattaaagacaccaagataactagctgcaaacatcccagacaagccgatcatactcgaaatcaaccaccttctctatcacatcatattgaatttaaggtttatccaatcacattcaaagttactaactacagcaaccaggaaaatcctcgaaagaattagaagcatcaatgaagaagcattgaacatcacttctataatccctccaggaaaccatcagaagtaaaacattctctggcccccattggttgaatttttttctaataagccaaaacggcttattagagaataaaaataaatgtgtaggtaaaacttttatatatgtgttttttttaacttaaaagccaatactgaaaaaaactacgttgaaaatatctcagaatcaatctcaaaattaagtttgaaaattcaaaatttggcttattctttggcttattgggccatctgatgggagcctctatatttggtagcacataactaaataaataaaagggttcatattcctaattgtcgaaagtctctcaaagtgacataacacactagtatttacactacggacaaccctcctagtttagactttagagttacatgttgcccagtttcgaggtaaagtagactagctcaaatgtgtctcaaacagtcgttcccggaacataagtcacaagtgttcccacgtgtaggcatgtttcacgcttagatcgatcgagtttcgtttccatctgtacgtacgttgtaccaccccaacccgtcgatcgatatgatcgatcgtcccctataaattggacatcctggacatgctctttcttcagacatctccacccttcctatcttgatatataacttcgacacactgacaacttaatttgtca

SEQ ID NO: 19 - DNA, 2096 bp, Oryza sativa
ccccgtctttatctactcgcgcccatcctctacttatctactcggcctctcctcctccactactcctcgcctggtcctcccctctcctcgatcccctcttccccttcccgggcctctccgtggtggttggcgcgcggcggcaggcggaggcgggcggtggtgcgcggcggcgtgacataagcggcgggctgatgggagggcgggcggcgggtggaggggaggatggtggcgggcggatgggtgggcgacaacgggtgggcggcagcgggaggcggtgccctcccttctgccggaggggaggcgggaggcggcagcggcagcggcggatttttttgttcttttttttttagtatttgtgattcattggatctgagatgtataatttgtgatgtatttttttttagaatttgtgatgtgaatctatgatttgtgatgttactttgatttggggatatgtagggagcaactcgatttggaaaaatcaaaatgcaagcagcaaagaaacaatgacaaaaaaagaagaaaaatggtgaccaaccgaaactgccgcaaccatcttttaatcccggttggtaacaccaaccagaactgaagatggacatctttaatttagtctcggattcacggtcctggtttacaacccgggactaaagggggttgcgaaccaggactaaagaagggttttccagcagttacagaaattaaaagtatatagtatttgctatatttactattttgccactatgtttgtcggagtgtgtacatagatagggggccattcgatgggaccgctttctatgagttcgggggtgtcgcacgtgagggagcgctcgggttcccattgaggacggaaacaaatgaacaaacaataattatattagtttgagacatccggatgtgtaacatcctactcctattgtgtcatgtgttttctctcttgctagtgaatgatcttatatgtcagacttagagtcgtagttcatctcttttggatttccgaagaacttcactgtgtatggctatatgttctaataactctgtatgttcgaacctcctttctcttggtcatggctctcccattatatctatctagggacaccacatgatcagaatattagggagatgctaattaagtgcatagtgccggattatgtaaggaaaatcttgtcagcactgctcctctctccctttttgcgaggcttagcgtagaaccacgaaaaaaaaacttgtattaaagacaccaagataactagctgcaaacatcccagacaagccgatcatactcgaaatcaaccaccttctctatcacatcatattgaatttaaggtttatccaatcacattcaaagttactaactacagcaaccaggaaaatcctcgaaagaattagaagcatcaatgaagaagcattgaacatcacttctataatccctccaggaaaccatcagaagtaaaacattctctggcccccattggttgaatttttttctaataagccaaaacggcttattagagaataaaaataaatgtgtaggtaaaacttttatatatgtgttttttttaacttaaaagccaatactgaaaaaaactacgttgaaaatatctcagaatcaatctcaaaattaagtttgaaaattcaaaatttggcttattctttggcttattgggccatctgatgggagcctctatatttggtagcacataactaaataaataaaagggttcatattcctaattgtcgaaagtctctcaaagtgacataacacactagtatttacactacggacaaccctcctagtttagactttagagttacatgttgcccagtttcgaggtaaagtagactagctcaaatgtgtctcaaacagtcgttcccggaacataagtcacaagtgttcccacgtgtaggcatgtttcacgcttagatcgatcgagtttcgtttccatctgtacgtacgttgtaccaccccaacccgtcgatcgatatgatcgatcgtcccctataaattggacatcctggacatgctctttcttcagacatctccacccttcctatcttgatatataacttcgacacactgacaacttaatttgtca

SEQ ID NO: 20 - DNA, 13274 bp, artificial
  misc_feature (1)..(13274): plasmid pNOV2347
ccaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcggggatctaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatggtcgacctcgaatcaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgttgattcgaggggatcctctagagtcgacctgcaggcatgcaaagctcggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacaagcttggcgcgccggtac

SEQ ID NO: 21 - DNA, 2001 bp, artificial
  misc_feature (1)..(2001): GIG sequence
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 22 - DNA, 12817 bp, artificial
  misc_feature (1)..(12817): plasmid pNOV6800
tttgtacaaacttgttgattcgaggggatcctctagagtcgacctgcaggcatgcaaagctcggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacaagcttggcgcgccggtacccaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcggggatctaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatggtcgacctcgaatcaaccactttgtacaagaaagctgggttgacaaattaagttgtcagtgtgtcgaagttatatatcaagataggaagggtggagatgtctgaagaaagagcatgtccaggatgtccaatttataggggacgatcgatcatatcgatcgacgggttggggtggtacaacgtacgtacagatggaaacgaaactcgatcgatctaagcgtgaaacatgcctacacgtgggaacacttgtgacttatgttccgggaacgactgtttgagacacatttgagctagtctactttacctcgaaactgggcaacatgtaactctaaagtctaaactaggagggttgtccgtagtgtaaatactagtgtgttatgtcactttgagagactttcgacaattaggaatatgaacccttttatttatttagttatgtgctaccaaatatagaggctcccatcagatggcccaataagccaaagaataagccaaattttgaattttcaaacttaattttgagattgattctgagatattttcaacgtagtttttttcagtattggcttttaagttaaaaaaaacacatatataaaagttttacctacacatttatttttattctctaataagccgttttggcttattagaaaaaaattcaaccaatgggggccagagaatgttttacttctgatggtttcctggagggattatagaagtgatgttcaatgcttcttcattgatgcttctaattctttcgaggattttcctggttgctgtagttagtaactttgaatgtgattggataaaccttaaattcaatatgatgtgatagagaaggtggttgatttcgagtatgatcggcttgtctgggatgtttgcagctagttatcttggtgtctttaatacaagtttttttttcgtggttctacgctaagcctcgcaaaaagggagagaggagcagtgctgacaagattttccttacataatccggcactatgcacttaattagcatctccctaatattctgatcatgtggtgtccctagatagatataatgggagagccatgaccaagagaaaggaggttcgaacatacagagttattagaacatatagccatacacagtgaagttcttcggaaatccaaaagagatgaactacgactctaagtctgacatataagatcattcactagcaagagagaaaacacatgacacaataggagtaggatgttacaagcctgcttt

(19) United States
(12) Patent Application Publication
(10) Pub. No.: US 2004/0103453 A1
(43) Pub. Date: May 27, 2004

(54) Lipoxygenase genes, promoters, transit peptides and proteins thereof

(76) Inventors: Robert Dudler, Zurich (CH); Ulrich Schaffrath, Aachen (DE); Kay Lawton, Research Triangle Park, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/332,406

(22) PCT Filed: Jul. 12, 2001
(86) PCT No.: PCT/EP01/08085

(30) Foreign Application Priority Data
Jul. 13, 2000 (GB) 0017275.9
Sep. 15, 2000 (GB) 0022739.7

(51) Int. Cl. A01H 1/00; C12N 15/82; C07H 21/04; C12N 9/02; C12N 5/04

(52) U.S. Cl. 800/279; 530/370; 435/189; 435/320.1; 435/419; 536/23.2

(57) ABSTRACT

This invention describes novel lipoxygenase genes and promoters, transit peptides and proteins derived therefrom. More specifically, this invention describes novel promoters that confer chemically inducible but not wound- or pathogen-inducible expression to an associated nucleotide sequence. The invention further describes peptides capable of targeting and associated protein to plastids and proteins having lipoxygenase activity which can be used to inhibit fungal mycotoxins. The invention also describes recombinant sequences containing such promoter sequences, and/or sequences encoding transit peptides and proteins according to the invention. The said recombinant DNA sequences may be used to create transgenic plants, but especially transgenic plants expressing a nucleotide sequence of interest in response to chemicals but not in response to wounding or pathogens.

The present invention relates to novel lipoxygenase genes and promoters, transit peptides and proteins derived therefrom. The present invention also relates the methods of using the novel lipoxygenase genes, promoters, transit peptides and proteins. The present invention also relates to isolated nucleic acid molecules encoding polypeptides having lipoxygenase activity and transit peptides. More specifically, this invention relates to isolated nucleic acid molecules encoding novel promoters that confer chemically inducible but not wound- or pathogen-inducible expression to an associated nucleotide sequence. Furthermore, the invention relates to peptides capable of targeting an associated protein to plastids. The invention also relates to proteins having lipoxygenase activity and to their use in inhibiting fungal mycotoxins. The invention further relates to recombinant nucleic acid molecules comprising nucleic acid molecules encoding the novel lipoxygenase genes, promoters or transit peptides. Also, the invention relates to host cells, plants or progeny thereof comprising the nucleic acid molecules or recombinant molecules described herein.

Plants are exposed to a variety of microbes during their life cycle, many of which are capable of causing disease. As a consequence plants have developed multiple defense strategies to avoid colonization. Certain treatments with chemical or biological agents can induce a normally susceptible plant to become systemically resistant to a subsequent inoculation with virulent pathogens. This phenomenon is known as systemic acquired resistance, or SAR. In rice, for example, treatment with the chemical N-cyanomethyl-2-chloroisonicotinamid, a derivative of 2,6-dichloroisonictinic acid (INA), has good resistance inducing activity against rice blast disease. Interestingly, treatment with N-cyanomethyl-2-chloroisonicotinamid, induced the enzyme lipoxygenase (LOX, linoleate:oxygen oxidoreductase, EC 1.13.11.12) (Seguchi et al. (1992, Journal Pest Sci. 17,107-113)), an enzyme known to be involved in plant defense against pathogens. Treatment with the rice blast fungus itself also induced this lipoxygenase. However, in modern agriculture, there is a desire to have a gene at hand, that is only induced by treatment with a chemical, but not by pathogens or wounding. Therefore, it is a major objective of the present invention to provide a lipoxygenase gene that is chemically induced, but not by pathogens or wounding.

It is another objective of the present invention, to provide the promoter and the transit peptide and protein encoded by such a lipoxygenase gene for use in agricultural biotechnology. In agricultural biotechnology, plants can be modified according to one's needs. One way to accomplish this is by using modern genetic engineering techniques. For example, by introducing a gene of interest into a plant, the plant can be specifically modified to express a desirable phenotypic trait. For this, plants are transformed most commonly with a heterologous gene comprising a promoter region, a coding region and a termination region. When genetically engineering a heterologous gene for expression in plants, the selection of a promoter is often a critical factor. While it may be desirable to express certain genes constitutively, i.e. throughout the plant at all times and in most tissues and organs, other genes are more desirably expressed only in response to particular stimuli or confined to specific cells or tissues. Chemically inducible promoters have been previously described (see, for example EP A-332 104). However, these promoters are also induced by pathogens. There are however occasions where it is desirable to use a promoter that is chemically induced but not by pathogens or wounding. Therefore, it is a major objective of the present invention to provide such alternative promoters for expression of a nucleotide sequence of interest in plants. The invention also provides recombinant DNA molecules, expression vectors and transgenic plants comprising the promoters of the present invention. When genes of interest are introduced into plants, they are most commonly expressed in the cytoplasm. Alternatively, one might wish to express those genes in other compartments of the cell. This can be accomplished, for example, by introducing the gene of interest into the plastid genome instead of the nuclear genome. However, currently, plastid transformation is not a routine procedure for all of the agriculturally important crops. Another possible way to express a protein of interest in plastids is to add a DNA sequence encoding a transit peptide to the 5′-end of the DNA sequence encoding a protein of interest and to express this DNA sequence from the nuclear genome. Transit peptides are peptides that are capable of targeting an associated protein to plastids. It is thus another objective of the present invention to provide such transit peptides. The invention also provides recombinant DNA molecules, expression vectors and transgenic plants comprising the transit peptides of the present invention. The transit peptides can be used in completely heterologous constructs or together with the promoter or coding region they are naturally associated with. The present invention also provides recombinant DNA molecules, expression vectors and transgenic plants comprising the transit peptides of the present invention.

In agricultural biotechnology not only the choice of the promoter is of importance, but also the choice of the associated DNA encoding a desirable phenotypic trait. A particularly desirable phenotypic trait is the lipoxygenase protein of the present invention. The invention thus provides recombinant DNA molecules, expression vectors and transgenic plants comprising the lipoxygenase protein of the present invention.

The present invention thus provides:

an isolated nucleic acid molecule capable of driving chemically inducible but not wound- or pathogen-inducible expression of an associated nucleotide sequence in particular, wherein said isolated nucleic acid molecule

is a component of the Pstl/Pstl fragment of about 4.5 kb in length from plasmid pBSK+LOX4A which has been deposited under accession no DSM 13524

is a component of the nucleotide sequence depicted in SEQ ID NO:17

is depicted in SEQ ID NO:18

is depicted in SEQ ID NO:19

comprises the nucleotide sequence depicted in SEQ ID NO:1

comprises the nucleotide sequence depicted in SEQ ID NO:2

comprises nt 1 to nt 1358 of the nucleotide sequence depicted in SEQ ID NO:2

comprises the nucleotide sequence depicted in SEQ ID NO:3

comprises nt 1702 to nt 2104 of SEQ ID NO:2 and/or nt 1 to nt 97 of SEQ ID NO:3 and/or nt 367 to nt 1283 of SEQ ID NO:3 of SEQ ID NO:3

comprises a combination of any one of the nucleotide sequences or portions thereof depicted in SEQ ID NO:1, SEQ ID NO:2 and SEQ ID NO:3

hybridizes under stringent conditions to SEQ ID NO:1, SEQ ID NO:2, SEQ ID NO:3, SEQ ID NO:17, SEQ ID NO:18 or SEQ ID NO:19, or to the 4.5 kb Pstl fragment of plasmid pBSK+LOX4A which has been deposited under accession no DSM 13524, wherein said nucleic acid molecule is capable of driving chemically inducible but not wound- or pathogen-inducible expression of an associated nucleotide sequence

comprises a consecutive stretch of at least 50 nt, preferably of about 500 bases, particularly of between about 1000 bases and about 1500 bases, more particularly of about 2000 bases and most particularly of between about 3000 bases and about 4500 bases in length of SEQ ID NO:1, SEQ ID NO:2, SEQ ID NO:3, SEQ ID NO:17, SEQ ID NO:18 or SEQ ID NO:19, or of the 4.5 kb Pstl fragment of plasmid pBSK+LOX4A which has been deposited under accession no DSM 13524, wherein said isolated nucleic acid molecule is capable of driving chemically inducible but not wound- or pathogen-inducible expression of an associated nucleotide sequence, in particular, wherein said consecutive stretch of at least 50 nt has at least 70%, preferably 80%, more preferably 90% and most preferably 95% sequence identity sequence identity with a consecutive stretch of corresponding length of SEQ ID NO:1, SEQ ID NO:2, SEQ ID NO:3, SEQ ID NO:17, SEQ ID NO:18 or SEQ ID NO:19, or the 4.5 kb Pstl fragment of plasmid pBSK+LOX4A which has been deposited under accession no DSM 13524

wherein the chemical inducer capable of inducing said nucleic acid molecule is selected from the group consisting of BTH (benzo(1,2,3)thiadiazole-7-carbothioic acid S-methyl ester), INA (2,6-dichloroisonicotinic acid) and probenazole

wherein the chemical inducer capable of inducing said nucleic acid molecule is jasmonic acid

Further provided are recombinant nucleic acid molecules comprising a nucleic acid molecule according to the invention operably linked to a nucleotide sequence of interest in particular, wherein

the nucleotide sequence of interest comprises a protein, polypeptide or peptide coding sequence

the coding sequence comprises at its 5′-end a nucleotide sequence encoding the amino acid sequence depicted in SEQ ID NO:6

the coding sequence encodes a desirable phenotypic trait

the coding sequence encodes a selectable or screenable marker gene

the coding sequence encodes a protein conferring antibiotic resistance, virus resistance, insect resistance, disease resistance, or resistance to other pests, herbicide tolerance, improved nutritional value, improved performance in an industrial process or altered reproductive capability

the coding sequence encodes commercially valuable enzymes or metabolites in the plant

the coding sequence is in antisense orientation

Further provided are isolated nucleic acid molecules expression vectors comprising an isolated nucleic acid molecule or a recombinant nucleic acid molecule of the invention as well as host cells stably transformed with a isolated nucleic acid molecule or a recombinant nucleic acid molecule according to the invention in particular, wherein

the host cell is a bacterium

the host cell is a plant cell

the host cell is a plant cell selected from the group consisting of rice, maize, wheat, barley, rye, sweet potato, sweet corn, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar-beet, sunflower, rapeseed, clover, tobacco, carrot, cotton, alfalfa, potato, eggplant, cucumber, Arabidopsis thaliana, and woody plants such as coniferous and deciduous trees, but particularly rice, maize, wheat, barley, cabbage, cauliflower, pepper, squash, melon, soybean, tomato, sugar-beet, sunflower or cotton, rice, maize, wheat, Sorghum bicolor, orchardgrass, sugar beet and soybean cells

the host cell is a plant cell from a dicotyledonous plant

the host cell is a plant cell from a dicotyledonous plant selected from the group consisting of soybean, cotton, tobacco, sugar beet and oilseed rape

the host cell is a plant cell from a monocotyledonous plant

the host cell is a plant cell from a monocotyledonous plant selected from the group consisting of maize, wheat, sorghum, rye, oats, turf grass, rice, and barley.

Further provided are plants and the progeny thereof stably transformed with a nucleic acid molecule or a recombinant nucleic acid molecule according to the invention. In particular, wherein said plant is selected from the group consisting of maize, wheat, sorghum, rye, oats, turf grass, rice, barley, soybean, cotton, tobacco, sugar beet and oilseed rape. Further provided are seeds from the transformed plants and progeny thereof.

In addition, use of the isolated nucleic acid molecule of the invention to express a nucleotide sequence of interest is provided.

The present invention further discloses

the use of the isolated nucleic acid molecule according to the invention to express a nucleotide sequence of interest

a method of producing an isolated nucleic acid molecule according to the invention, wherein the nucleic acid molecule is produced by a polymerase chain reaction wherein at least one oligonucleotide used comprises a sequence of nucleotides which represents a consecutive stretch of 15 or more base pairs of SEQ ID NO:1, SEQ ID NO:2, SEQ ID NO:3, SEQ ID NO:17, SEQ ID NO:18 or SEQ ID NO:19.

The invention also provides isolated nucleic acid molecules encoding the amino acid sequence depicted in SEQ ID NO:6, wherein said amino acid sequence is capable of targeting an associated protein to plastids in particular, wherein

said nucleotide sequence is the sequence depicted in SEQ ID NO:4

said nucleotide sequence hybridizes under stringent conditions to SEQ ID NO:4 in particular, wherein said sequence has 70%, preferably 80%, more preferably 90% sequence identity with the nucleotide sequence of SEQ ID NO:4 and the encoded peptide is capable of targeting an associated protein to plastids Further are provided polypeptides or peptides encoded by the isolated nucleic acid molecules described above as well as the use of said polypeptides or peptides to target an associated protein of interest to plastids. In addition, the invention provides isolated nucleic acid molecules which hybridize under stringent conditions to SEQ ID NO:5, and wherein the protein encoded by said nucleic acid molecule has at least 65%, preferably 75% more preferably 85% and most preferably 95% amino acid sequence identity with the amino acid sequence depicted in SEQ ID NO:7 and encodes a protein with lipoxygenase activity. The invention further provides nucleic acid molecules as mentioned hereinbefore, wherein said nucleic acid molecules encode the protein depicted in SEQ ID NO:7. Further are provided proteins encoded by said nucleic acid molecules described hereinbefore, in particular, SEQ ID NO:7 or portions of the proteins or polypeptides having lipoxygenase activity. The invention further discloses the use of the protein as mentioned hereinbefore to inhibit fungal mycotoxins, in particular aflatoxins. The invention further provides methods of increasing plant disease resistance or inhibiting fungal mycotoxins by expressing the isolated nucleic acid molecules of the present invention that encode lipoxygenase activity in transformed plants. Further provided are recombinant nucleic acid molecule comprising the nucleic acid molecules as described above, host cells stably transformed therewith, in particular wherein said host cell is a plant cell and plants and the progeny thereof stably transformed with a recombinant nucleic acid molecule as described above. In order to ensure a clear and consistent understanding of the specification and the claims, the following definitions are provided:

DNA shuffling: DNA shuffling is a method to rapidly, easily and efficiently introduce rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule.

Expression: refers to the transcription and/or translation of an endogenous gene or a transgene in plants. In the case of antisense constructs, for example, expression may refer to the transcription of the antisense DNA only.

Functionally equivalent sequence: refers to a DNA sequence which has promoter activity substantially similar to the rice lipoxygenase gene promoter or parts thereof and which under stringent hybridizing conditions hybridizes with the said promoter sequences.

Gene: refers to a coding sequence and associated regulatory sequence wherein the coding sequence is transcribed into RNA such as mRNA, rRNA, tRNA, snRNA, sense RNA or antisense RNA. Examples of regulatory sequences are promoter sequences, 5′- and 3′-untranslated sequences and termination sequences. Further elements that may be present are, for example, introns.

Gene of interest: refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

Heterologous as used herein means of different natural or of synthetic origin. For example, if a host cell is transformed with a nucleic acid sequence that does not occur in the untransformed host cell, that nucleic acid sequence is said to be heterologous with respect to the host cell. The transforming nucleic acid may comprise a heterologous promoter, heterologous coding sequence, or heterologous termination sequence. Alternatively, the transforming nucleic acid may be completely heterologous or may comprise any possible combination of heterologous and endogenous nucleic acid sequences.

Leader region: region in a gene between transcription start site and translation start site.

LOX: lipoxygenase.

Marker gene: refers to a gene encoding a selectable or screenable trait.

nt: nucleotide, and are naturally occurring or synthetic nucleotides.

Nucleic acid molecule: is any single or double stranded polynucleotide that is commonly either DNA or RNA, and can comprise naturally occurring or synthetic nucleotides.

Operably linked to/associated with: a regulatory DNA sequence is said to be “operably linked to” or “associated with” a DNA sequence that codes for an RNA or a protein if the two sequences are situated such that the regulatory DNA sequence affects expression of the coding DNA sequence.

Plant: refers to any plant, particularly to seed plants.

Plant cell: structural and physiological unit of the plant, comprising a protoplast and a cell wall. The plant cell may be in form of an isolated single cell or a cultured cell, or as a part of higher organized unit such as, for example, a plant tissue, or a plant organ.

Plant material: refers to leaves, stems, roots, flowers or flower parts, fruits, pollen, pollen tubes, ovules, embryo sacs, egg cells, zygotes, embryos, seeds, cuttings, cell or tissue cultures, or any other part or product of a plant

Polynucleotide: any single-stranded homo-or heteropolymer of at least about ten nucleotides connected by phosphodiester linkages between (usually) the 3′ position of the glycose moiety of one nucleotide and the 5′ position on the glycose moiety of the adjacent nucleotide, or any double-stranded molecule comprised of two such single-stranded molecules held together by hydrogen bonds.

Promoter: refers to a DNA sequence that initiates transcription of an associated DNA sequence. The promoter region may also include elements that act as regulators of gene expression such as activators, enhancers, and/or repressors and may include all or part of the 5′ non-translated region.

Protein, Polypeptide or peptide: are used herein interchangeably and are amino acid residues connected by peptide linkages.

Recombinant DNA molecule: a combination of DNA sequences that are joined together using recombinant DNA technology.

Recombinant DNA technology: procedures used to join together DNA sequences as described, for example, in Sambrook et al., 1989, Cold Spring Harbor, N.Y.: Cold Spring Harbor Laboratory Press.

Screenable marker gene: refers to a gene whose expression does not confer a selective advantage to a transformed cell, but whose expression makes the transformed cell phenotypically distinct from untransformed cells.

Selectable marker gene: refers to a gene whose expression in a plant cell gives the cell a selective advantage. The selective advantage possessed by the cells transformed with the selectable marker gene may be due to their ability to grow in the presence of a negative selective agent, such as an antibiotic or a herbicide, compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a plant cell in the presence of the selective agent, compared to the absence of the selective agent, has a positive effect on the transformed plant cell and a negative effect on the un-transformed plant cell, for example with respect to growth, and thus gives the transformed plant cell a positive selective advantage.

Sequence identity: the percentage of sequence identity is determined using computer programs that are based on dynamic programming algorithms. Computer programs that are preferred within the scope of the present invention include the BLAST (Basic Local Alignment Search Tool) search programs designed to explore all of the available sequence databases regardless of whether the query is protein or DNA. Version BLAST 2.0 (Gapped BLAST) of this search tool has been made publicly available on the Internet (currently http://www.ncbi.nim.nih.gov/BLAST/). It uses a heuristic algorithm, which seeks local as opposed to global alignments and is therefore able to detect relationships among sequences, which share only isolated regions. The scores assigned in a BLAST search have a well-defined statistical interpretation. Said programs are preferably run with optional parameters set to the default values.

Transformation: refers to the introduction of a nucleic acid into a cell. In particular, it refers to the stable integration of a DNA molecule into the genome of an organism of interest.

The present invention relates to lipoxygenase genes, and to promoters, transit peptides and proteins derived therefrom. Preferred are lipoxygenase genes that are chemically induced, but not by pathogens or wounding. In particular, said lipoxygenase genes are from rice. Such lipoxygenase genes, or portions or fragments therefrom, can be obtained, for example, by a PCR-based strategy. For this, known lipoxygenase coding sequences, for example, from rice (Peng et al. (1994) J. Biol. Chem. 269, 3755-3761; Ohta et al. (1992) Eur. J. Biochem. 206, 331-336) and from wheat (Gorlach et al. (1996) Plant Cell. 8, 629-643) are aligned to identify conserved regions using computer programs known in the art. By this method, several conserved regions are identified, one of which is near the C-terminus and contains the amino acid sequence HAAVNFG that is invariant in all three sequences. Then, total RNA or polyA⁺ RNA is isolated from untreated control leaves and from leaves sprayed with a 100 ppm INA solution and harvested 24 and 48 hours after treatment. The RNA samples are used as templates for RT-PCR using the degenerate oligonucleotide 5′-CAYGCNGTNAANTTYGG-3′ (SEQ ID NO:8), which corresponds to the HAAVNFG amino acid sequence motif in the C-terminal region of the rice RLL2 lipoxygenase (Peng et al. (1994) J. Biol. Chem. 269, 3755-3761), as the forward primer and an anchored oligo-dT primer as the reverse primer (5′-AATGCTTTTTTTTTTTTTTTV-3′, SEQ ID NO:9). When this method is performed with total RNA or polyA⁺ RNA from rice, a PCR product of approximately 600 bp arises on ethidiumbromide stained agarose gels only in the INA-treated sample but not in the control. Those of skill in the art know that the size of the band can be smaller or larger, depending on the organism from which the RNA is isolated. The obtained band can be cloned and sequenced and used as a probe to screen cDNA or genomic libraries to obtain full-length lipoxygenase cDNA or genomic clones by methods known in the art. Upon screening a rice cDNA library constructed from INA-treated leaves, a full-length rice lipoxygenase cDNA clone of 3018 bp in length is obtained (SEQ ID NO:5). This cDNA clone, designated RCI-1 (rice chemically induced cDNA 1), contains an open reading frame of 2766 bp (from base 48 to base 2816 of SEQ ID NO:5) encoding a protein of 922 amino acid residues with a predicted Mr of 105 kDa (SEQ ID NO:7). To those with skill in the art it is known that the obtained cDNA clone can be larger or smaller, depending on whether the clone is full-length or not, on the length of the 5′ and 3′ untranslated region, and on the organism from which the library is constructed. When the RCI-1 cDNA is used as a probe in Northern blot analyses with RNA from chemically treated leaves, such as leaves treated with INA, BTH, probenazole or jasmonic acid, a strong hybridization signal is observed, indicating the accumulation of RCI-1 mRNA. No such mRNA accumulation is observed when RNA from wounded or pathogen-treated leaves is used. This is surprising, as wounding is known to increase endogenous levels of jasmonic acid in rice and induces increased systemic protection against rice blast infection (Schweizer et al. (1998) Plant J. 14, 475-481; Schweizer et al. (1997) Plant Physiol. 114, 79-88). The lipoxygenase of the present invention however is not induced by pathogens such as the rice blast fungus Magnaporthe grisea nor the bacterial pathogen Pseudomonas syringae pv. syringae. This indicates that the promoter region of the corresponding gene must contain regulatory elements that confer a chemically, but not wound- or pathogen inducible expression pattern to an associated coding sequence. The protein encoded by the RCI-1 cDNA is most similar to the barley LOX2:Hv:1 (60% identity and 68% similarity). Sequence identity (similarity) at the amino acid level are 43% (52%) for the rice lipoxygenase L-2 predominately found in kernels and seedlings (Ohta et al. (1992) Eur. J. Biochem. 206, 331-336) and 50% (58%) for the Magnaporthe grisea-induced rice lipoxygenase RLL2 (Peng et al. (1994) J. Biol. Chem. 269, 3755-3761). DNA sequences embraced by the present invention are those that hybridize to the RCI-1 cDNA clone (SEQ ID NO:5) under stringent conditions and whose coding sequences have at least 65%, preferably 75%, more preferably 85% and most preferably 95% amino acid sequence identity to the protein depicted in SEQ ID NO:7 and encode a protein with lipoxygenase activity. The lipoxygenase cDNA of the present invention can be expressed in E. coli or in any other expression system suitable to express eukaroytic sequences by methods known in the art. The expressed protein is then analyzed and, optionally, purified. All these methods are known to a person skilled in the art. When an extract of E. coli cells expressing a cDNA of the present invention is analyzed, increased LOX activity using linoleic acid as a substrate is detected, while control extracts of E. coli without expression construct or containing the empty vector do not have detectable LOX activity. Maximal activity is observed around pH 8 to 9, indicating that RCI-1 must be classified as a type 1 LOX (Siedow (1991) Ann. Rev. Plant Physiol. Plant Mol. Biol. 42, 145-188). However, it should be noted that recently a second classification based on the presence of a plastomic transit peptide was introduced (Shibata et al. (1994) Plant Mol. Biol. Rep. 12, 41-42). According to this scheme, RCI-1 must be classified as a type 2 LOX. When the reaction products of the recombinant lipoxygenase of the present invention are analyzed by HPLC (Bohland et al. (1997) Plant Physiol. 114, 679-685), (13S)-hydroperoxy-(9Z, 11E, 15Z)-octadecatrienoic acid (13-HPOD) is the predominant product, irrespective of whether linoleic acid or linolenic acid serves as a substrate for the enzyme. (9S)-hydroperoxy-(10E, 12Z, 15Z) octadecatrienoic acid (9-HPOD) is only detected in minor amounts. Reaction products derived from 13-HPOD have been reported to act as antimicrobial substances against Magnaporthe grisea (Shimura et al., (1981) Agric Biol Chem 45:1431-1435; Shimura et al (1983) Agric Biol Chem 47:1983-1989). This indicates that the lipoxygenase of the present invention, in particular RCI-1 LOX, is involved in the generation of fatty acid derivatives that may act as signals or exhibit direct antimicrobial activity (reviewed in Slusarenko, A. J. (1996) The role of lipoxygenase in resistance to infection. In Lipoxygenase and Lipoxygenase Pathway Enzymes (Piazza, G. J., ed) pp.176-197. American Oil Chemists Society Press, Champaign, Ill.). In one particular embodiment, the lipoxygenase of the present invention is suited to eliminate or substantially reduce the activity of fungal mycotoxins, which include, but are not limited to aflatoxins and their precursor sterigmatocystin, citrinin, fungal tremorgens, lupinosis, ochratoxins, patulin, rubratoxins, sporidesmin, stachybotyrotoxins, trichothecens and zearalenone, but particularly aflatoxin and sterigmatocystin. An alignment of the amino acid sequence of the lipoxygenase protein of the present invention with the amino acid sequence of other lipoxygenases such as barley LoxA (GenBank accession number L35931) or rice L-2 (Swissprot accession number P29250) shows that the lipoxygenase of the present invention has a N-terminal extension of between 30 and 50 amino acid residues. In the case of the rice lipoxygenase RCI-1 that extension is about 47 amino acids in length. This N-terminal extension clearly separates this class of LOX species from another class that is predominately found in kernels and seedlings and that includes LoxA, LoxB, and LoxC from barley (van Mechelen et al. (1999) Plant Mol. Biol. 39,1283-1298), and LOX L-2 from rice (Ohta et al (1992) Eur. J. Biochem. 206, 331-336). When parts or all of this N-terminal extension is fused to the N-terminal region of a reporter gene, the reporter gene is targeted to plastids, in particular to chloroplasts. For example, when a chimeric gene is constructed with the first 158 bp of the RCI-1 cDNA (SEQ ID NO:4) fused to the 5′ end of the coding sequence of the green fluorescent protein (GFP), a modified GFP is obtained which contains at its N-terminus the first 37 amino acids of RCI-1 (SEQ ID NO:6). When said construct is introduced into Arabidopsis tissue, for example, by Agrobacterium based transformation system, a strict congruence of GFP fluorescence and chlorophyll autofluorescence is observed, indicating that the fusion protein is localized in the chloroplasts. Thus, the N-terminal extensions of the lipoxygenase proteins of the present invention function as transit peptides to transfer associated proteins to plastids, particularly to chloroplasts.

In addition, the present invention also provides promoters capable of conferring chemically inducible, but not wound- or pathogen-inducible expression to an associated nucleotide sequence of interest. Preferred are promoter sequences obtainable from the rice lipoxygenase gene RCI-1. Nucleotide sequences comprising functional and/or structural equivalents thereof are also embraced by the invention. The present invention thus relates to nucleotide sequences that function as promoters of transcription of associated nucleotide sequences. The promoter region may also include elements that act as regulators of gene expression such as activators, enhancers, and/or repressors and may include the 5′ non-translated leader sequence of the transcribed mRNA and/or introns and, optionally, exons. Chemically inducible, but not wound- or pathogen inducible expression means that the nucleotide sequence of interest is preferentially expressed when a chemical compound according to the invention is applied, but not upon wounding or exposure to pathogens. Thus, the nucleotide sequence according to the invention is useful for chemically inducible, but not wound- or pathogen inducible expression of an associated nucleotide sequence of interest, which preferably is a coding sequence. It is known to the skilled artisan that the associated coding sequence of interest can be expressed in sense or in antisense orientation. Further, the coding sequence of interest may be of heterologous or homologous origin with respect to the plant to be transformed. In case of a homologous coding sequence, the nucleotide sequence according to the invention is useful for ectopic expression of said sequence. In one particular embodiment of the invention expression of the coding sequence of interest under control of a nucleotide sequence according to the invention suppresses its own expression and that of the original copy of the gene by a process called co-suppression.

The promoters of the present invention can be obtained, for example, from rice genomic DNA by probing a rice genomic library with a cDNA according to the invention using methods known in the art. It is obvious to a person skilled in the art that genomic DNA from any other organism, particularly from plants, can be used to obtain a lipoxygenase promoter from any organism of interest. This genomic DNA is then sequenced and aligned to the cDNA sequence. Basically, all nucleotide sequences upstream of the start codon are considered to be part of the lipoxygenase promoter region. In addition, introns and, optionally, exons can be added to this region to form a functional promoter that confers chemically inducible, but not wound- or pathogen inducible expression to an associated coding region. In a preferred embodiment of the invention, the lipoxygenase promoter is a component of the Pstl/Pstl fragment of about 4.5 kb in length from plasmid pBSK+LOX4A which has been deposited under accession no DSM 13524. SEQ ID NO:17 depicts the nucleotide sequence of the about 4.5 kb Pstl/Pstl fragment from plasmid pBSK+LOX4A. Other preferred embodiments of the invention are the nucleotide sequences depicted in SEQ ID Nos:18,19, 1, 2 and 3, which are components of the 4.5 kb Pstl/Pstl fragment as mentioned hereinbefore. Another preferred embodiment of the invention comprises nt 1 to nt 1358 of the nucleotide sequence depicted in SEQ ID NO:2. SEQ ID NO:1 comprises the 5′-end of the 4.5 kb Pstl/Pstl fragment. This nucleotide sequence is 358 nucleotides in length and contains at its 5′ end in position 1 to 6 the Pstl-site. The region between SEQ ID NO:1 and SEQ ID NO:2 of the 4.5 kb Pstl/Pstl fragment is between about 240 and 440 bp in length. The central region of the 4.5 kb Pstl/Pstl fragment is shown in SEQ ID NO:2 and is 2104 bp in length. It contains the putative TATA box (position 1261 to 1266 SEQ ID NO:2), the putative start codon (position 1359 to 1361 of SEQ ID NO:2), as well as the 5′ untranslated region and nucleotide sequences upstream of the putative TATA box. Comparison of the genomic DNA (SEQ ID NO:2) and the cDNA (SEQ ID NO:5) shows that the sequences located at position 1312 to 1701 of SEQ ID NO:2 comprise all or part of exon 1, and the sequences located at position 1702 to 2104 of SEQ ID NO:2 are the 5′ part of intron 1. The region between SEQ ID NO:2 and SEQ ID NO:3 of the 4.5 kb Pstl/Pstl fragment is between about 85 and 285 bp in length. The 3′ end of the 4.5 kb Pstl/Pstl fragment is shown in SEQ ID NO:3. This sequence depicts a nucleotide sequence of 1516 bp in length. It contains, in a 5′ to 3′ direction, the 3′ end of intron 1 (position 1 to 97 of SEQ ID NO:3) followed by exon 2 (position 98 to 366 of SEQ ID NO:3), intron 2 (position 367 to 1283 of SEQ ID NO:3) and part of exon 3 (position 1284 to 1516 of SEQ ID NO:3). The Pstl site is located at position 1511 to 1516.

Based on the sequence information given in SEQ ID NOs:1 to 3, the DNA sequences of the invention can be obtained, for example, by PCR using plasmid pBSK+LOX4A or genomic DNA from rice or any other organism of interest as template. The person skilled in the art knows how to arrive at such sequences using methods known in the art. These sequences then can be fused to reporter genes to demonstrate promoter activity. For example, chimeric genes can be constructed that include part of the 5′ regulatory sequence of the RCI-1 gene fused to the GFP coding sequence. To this end, pBSK+LOX4A (see Example 9) can be used as template for the polymerase chain reaction (PCR). Gene-specific primers can be designed to amplify the 5′ promoter region of the gene. Using combinations of, for example, the reverse primer R1 (SEQ ID NO:12) with forward primers F1 (SEQ ID NO:13) and F2 (SEQ ID NO:14) the regulatory sequences that are ˜1.2 kb and ˜2 kb upstream of the initiating methionine are isolated. The nucleotide sequence of the PCR fragment amplified with forward primer F1 and reverse primer R1 is shown in SEQ ID NO:18, and the nucleotide sequence of the PCR fragment amplified with forward primer F2 and reverse primer R1 is shown in SEQ ID NO:1 9. For ease of cloning the primers consist, for example, of gene specific sequences and attB recombination sites for the GATEWAY™ cloning technology (Life Technologies, GIBCO BRL, Rockville, Md. USA). As reverse primer, primer R1 can be used, which has the following sequence: 5′-CAAGAAAGCTGGGTTGACAAATTAAGTTGTCAGTGTG-3′ (SEQ ID NO:12). The gene specific sequence of reverse primer R1 is underlined (corresponds to position 1356 to 1334 of SEQ ID NO:2), the attB recombination sequence is denoted in italics. Examples for forward primers are the primers F1 and F2. Forward primer F1 has the following sequence: 5′-CAAAAAAGCAGGCTTGTAACATCCTACTCCTATTGTG-3′ (SEQ ID NO:13). The gene specific sequence of forward primer F1 is underlined (corresponds to bases 159 to 181 of SEQ ID NO:2), the attB recombination sequence is denoted in italics. F1 in combination with R1 amplifies a fragment of ˜1.2 kb. Forward primer F2 has the following sequence: 5′-CAAAAAAGCAGGCTCCCCGTCTTTATCTACTC-3′ (SEQ ID NO:14). The gene specific sequence of forward primer F2 is underlined (corresponds to bases 31 to 48 of SEQ ID NO: 1), the attB recombination sequence is denoted in italics. Primer F2 in combination with primer R1 amplifies a fragment of ˜2 kb. Using a nested PCR strategy the regulatory sequence can be amplified first with primers F1+R1 or F2+R1 followed by a second PCR with primer attB1 (5′-GGGGACAAGTTTGTACAAAAAAGCAGGCT-3′, SEQ ID NO:15) and primer attB2 (5′-GGGGACCACTTTGTACAAGAAAGCTGGGT-3′, SEQ ID NO:16). Optimal annealing temperatures can be determined using a gradient thermocyler (DNA Engine, MJ Research, Inc. Waltham, Mass. USA) and the following PCR conditions with gene-specific primers F1 +R1 or F2+R1: [(94° C.:10 sec):(94° C.:10 sec/45° C. to 70° C. gradient:10 sec/72° C.:10 sec)X15]. Following PCR the products can be visualized by gel electrophoresis, and DNA from the reaction with the highest Tm giving visible product can be selected for amplification with the attBl+attB2 primers. In the subsequent PCR amplification, the following PCR conditions can be used: [(94° C.:10 sec):(94° C.:10 sec/50° C. to 70° C. gradient 10 sec/72° C.:10 sec)X15). The resulting PCR product are then flanked by attB recombination sites which can be used to generate Entry Clones in pENTR via the BP reaction according to manufacturers protocol (see: Instruction Manual of GATEWAY™ Cloning Technology, GIBCO BRL, Rockville, Md. USA, http://www.lifetech.com/). The resulting plasmids contain ˜1.2 kb and ˜2 kb 5′ of the RCI-1 initiation codon and are referred to as pENTR+LOXp1.2, pENTR+LOXp2. The regulatory/promoter sequence is then fused to the mGFP-5 reporter gene (MRC Laboratory of Molecular Biology, Cambridge, England) by recombination using GATEWAY™ Technology according to manufacturers protocol as described in the Instruction Manual (GATEWAY™ Cloning Technology, GIBCO BRL, Rockville, Md. http://www.lifetech.com/). Briefly, according to this protocol the promoter fragment in the entry vector is recombined via the LR reaction with a binary Agrobacterium destination vector containing the mGFP-5 coding region that has an attR site 5′ to the GFP reporter. The orientation of the inserted fragment is maintained by the att sequences and the final construct is verified by sequencing. The construct is designated pLOXp1.2 promoter::GFP or pLOXp2promoter::GFP and can be transformed into Agrobacterium tumefaciens strains by electroporation. Any other binary vector can also be modified to accommodate promoter fragments of the invention to drive expression of an associated reporter gene. The skilled artisan knows how to construct such vectors starting from commercially available binary vectors such as, for example pGPTV-BLEO (ATCC number 77392), pBl 121 (Clontech, Palo Alto, Calif.), or pCambia 1302 (Cambia, Canberra, Australia). Transgenic plants are then produced using, for example, Agrobacterium-mediated transformation techniques. Expression of the gene fusion protein can be monitored in transformants by confocal imaging using a Leica-TCS confocal laser scanning microscope and a PLAPO×100 oil immersion objective (Leica Microsystems, Heidelberg, Germany) with the following filter settings: excitation 476/488 nm; GFP-emission 515-552 nm, chlorophyll-emission 673-695 nm. GFP fluorescence and chlorophyll autofluorescence are recorded simultaneously using independent 2-channel-detection. Confocal imaging of leaves from transgenic rice plants expressing the pRCI promoter::GFP construct can be carried out to assay promoter activity in response to abiotic and biotic inducers.

It is apparent to the skilled artisan that, based on the nucleotide sequences shown in SEQ ID NO:1 to 3, any primer combination of interest can be chosen to PCR amplify DNA fragments of various lengths that can be used according to the invention. Thus, any region of interest can be amplified from SEQ ID NOs:1 to 3. For example, primers can be designed to specifically amplify intron 1 or intron 2 or the 5′ upstream region. The 5′ upstream region is defined herein as the region between the putative TATA box and the putative start codon of the lipoxygenase protein. The skilled artisan also will consider to combine intron 1 and/or intron 2 with various parts of SEQ ID NOs:1, 2 and/or 3, such as to arrive at an DNA molecule comprising nt 1702 to nt 2104 of SEQ ID NO:2 and/or nt 1 to nt 97 of SEQ ID NO:3 and/or nt 367 to nt 1283 of SEQ ID NO:3 of SEQ ID NO:3. Further, it might also be desirable to combine any of these sequences with the 3′ untranslated region of the lipoxygenase cDNA sequence (position 2817 to 3018 of SEQ ID NO:5). The invention thus includes fragments derived from the rice RCI-1 lipoxygenase gene that function according to the invention, i.e. are capable of conferring chemically induced but not wound- or pathogen induced expression of an associated nucleotide sequence. This can be tested by generating such promoter fragments, fusing them to a selectable or screenable marker gene and assaying the fusion constructs for retention of promoter activity in transient expression assays with protoplasts or in stably transformed plants. Such assays are within the skill of the ordinary artisan. Preferred nucleic acid molecule fragments of the invention are of at least about 500 bases, particularly of between about 1000 bases and about 1500 bases, more particularly of about 2000 bases and most particularly of between about 3000 bases and about 4500 bases in length. It is also clear to the skilled artisan that mutations, insertions, deletions and/or substitutions of one or more nucleotides can be introduced into the nucleotide sequences of SEQ ID NOs:1, 2 and 3 or longer or shorter fragments derived from the sequence information thereof using methods known in the art. In addition, an unmodified or modified nucleotide sequence of the present invention can be varied by shuffling the sequence of the invention. To test for a function of variant nucleotide sequences according to the invention, the sequence of interest is operably linked to a selectable or screenable marker gene and expression of the marker gene is tested in transient expression assays with protoplasts or in stably transformed plants. It is known to the skilled artisan that nucleotide sequences capable of driving expression of an associated nucleotide sequence are build in a modular way. Accordingly, expression levels from shorter nucleic acid molecule fragments may be different than the one from the longest fragment and may be different from each other. For example, deletion of a down-regulating upstream element will lead to an increase in the expression levels of the associated nucleotide sequence while deletion of an up-regulating element will decrease the expression levels of the associated nucleotide sequence. Another way of identifying promoter elements necessary for regulated expression of an associated nucleotide sequence is the so-called linker-scanning analysis. Linker-scanning mutagenesis allows for the identification of short defined motifs whose mutation alters the promoter activity. Accordingly, a set of linker-scanning mutant promoters fused to the coding sequence of the GUS reporter gene or another marker gene can be constructed using methods known in the art. These construct are then transformed into Arabidopsis, for example, and GUS activity is assayed in several independent transgenic lines. The effect of each mutation on promoter activity is then compared to an equivalent number of transgenic lines containing an unmutated rice lipoxygenase gene promoter. It is expected, that when a motif is mutated that is involved in chemically, but not wound or pathogen-inducible expression, that the level of expression of the reporter gene is modified. If, for example, a higher average induction of GUS activity by a chemical inducer is detected than the one from the control construct most likely a negative regulatory element had been mutated in this construct. If, on the other hand, a complete loss of inducibility of GUS activity by a chemical regulator according to the invention is observed, most likely a positive regulatory element necessary chemical induction has been mutated. In a next step, particularly in the case of the putative positive regulatory element, the wild-type sequences corresponding to the mutated fragments are fused to a minimal promoter and the newly created promoter is tested for the ability to confer regulated expression to an associated marker gene.

Embraced by the present invention are also functional equivalents of the RCI-1 promoters of the present invention, i.e. nucleotide sequences that hybridize under stringent conditions to any one of SEQ ID NO:1, SEQ ID NO:2, SEQ ID NO:3, SEQ ID NO:17, SEQ ID NO:18 or SEQ ID NO:19, or to the 4.5 Pstl/Pstl fragment of plasmid pBSK+LOX4A which has been deposited under accession no DSM 13524. A stringent hybridization is performed at a temperature of 65° C., preferably 60° C. and most preferably 55° C. in double strength (2×) citrate buffered saline (SSC) containing 0.1% SDS followed by rinsing of the support at the same temperature but with a buffer having a reduced SSC concentration. Such reduced concentration buffers are typically one tenth strength SSC (0.1×SSC) containing 0.1% SDS, preferably 0.2×SSC containing 0.1% SSC and most preferably half strength SSC (0.5×SSC) containing 0.1% SDS. In fact, functional equivalents to all or part of the RCI-1 lipoxygenase promoter from other organisms can be found by hybridizing any one of SEQ ID NO:1, SEQ ID NO:2, or SEQ ID NO:3 or the 4.5 Pstl/Pstl fragment of plasmid pBSK+LOX4A which has been deposited under accession no DSM 13524 with genomic DNA isolated from an organism of interest, particularly from another monocot. The skilled artisan knows how to proceed to find such sequences as there are many ways known in the art to identify homologous sequences from other organisms. Such newly identified DNA molecules then can be sequenced and the sequence can be compared to any one of SEQ ID NO:1, SEQ ID NO:2, SEQ ID NO:3, SEQ ID NO:17, SEQ ID NO:18 or SEQ ID NO:19, or to the nucleotide sequence of the 4.5 Pstl/Pstl fragment of pBSK+LOX4A which has been deposited under accession no DSM 13524, and tested for promoter activity. Within the scope of the present invention are DNA molecules having at least 75%, preferably 80%, more preferably 90%, and most preferably 95% sequence identity to the nucleotide sequence of any one of SEQ ID NOs:1, 2, or 3 over a length of at least 50 nucleotides. The percentage of sequence identity is determined using computer programs that are based on dynamic programming algorithms. Computer programs that are preferred within the scope of the present invention include the BLAST (Basic Local Alignment Search Tool) search programs designed to explore all of the available sequence databases regardless of whether the query is protein or DNA. Version BLAST 2.0 (Gapped BLAST) of this search tool has been made publicly available on the Internet (currently http://www.ncbi.nlm.nih.gov/BLAST/). It uses a heuristic algorithm which seeks local as opposed to global alignments and is therefore able to detect relationships among sequences which share only isolated regions. The scores assigned in a BLAST search have a well-defined statistical interpretation. Said programs are preferably run with optional parameters set to the default values.

If desired, the promoters of the present invention can be fused with the nucleotide sequence encoding a transit peptide according to the invention for example, by using the nucleotide sequence depicted in SEQ ID NO:4, for chemically regulated expression of an associated coding region of interest in plastids, particularly in chloroplasts.

A chemical regulator according to the invention is defined as a substance which regulates expression of a gene through a chemically regulatable DNA sequence. The substance, in ionic or neutral form, with or without solvating or other complexing molecules or anions, will usually be exogenous relative to the system containing the chemically regulatable gene at the time regulation is desired. The use of exogenous chemical regulators is preferred because of the ease and convenience of controlling the amount of regulator in the system. However, the invention also includes the use of endogenous regulators, e.g., chemicals whose activities or levels in the system are artificially controlled by other components in, or acting on, the system.

Chemical regulators according to the invention include benzoic acid, salicylic acid, polyacrylic acid and substituted derivatives thereof; suitable substituents include lower alkyl, lower alkoxy, lower alkylthio and halogen, but particularly INA, BTH, probenazole, jasmonate, and methyl jasmonate. An additional group of regulators for the chemically regulatable DNA sequences and chimeric genes of this invention is based on the benzo-1,2,3-thiadiazole structure and includes, but is not limited to, the following types of compounds: benzo-1,2,3-thiadiazolecarboxylic acid, benzo-1,2,3-thiadiazolethiocarboxylic acid, cyanobenzo-1,2,3-thiadiazole, benzo-1,2,3-thiadiazolecarboxylic acid amide, benzo-1,2,3-thiadiazolecarboxylic acid hydrazide, and derivatives thereof. A preferred group of regulators includes, but is not limited to, benzo-1,2,3-thiadiazole-7-carboxylic acid, benzo-1,2,3-thiadiazole-7-thiocarboxylic acid, 7-cyanobenzo-1,2,3-thiadiazole, benzo-1,2,3-thiadiazole-7-carboxylic acid amide, benzo-1,2,3-thiadiazole-7-carboxylic acid hydrazide, and derivatives thereof. Suitable derivatives encompass but are not limited to representatives of said types of compounds wherein the benzo-1,2,3-thiadiazole moiety is unsubstituted or substituted by small substituents normally used in aromatic ring systems of agrochemicals such as lower alkyl, lower alkoxy, lower haloalkyl, lower haloalkoxy, lower alkylthio, cyano, nitro and halogen. Suitable derivatives further encompass, but are not limited to, representatives of said benzo-1,2,3-thiadiazole compounds wherein either the carboxylic acid, the thiocarboxylic acid, the carboxylic acid amide or the carboxylic acid hydrazide functional group is unsubstituted or substituted by aliphatic, araliphatic or aromatic residues. Suitable residues encompass, but are not limited to, alkyl (especially lower alkyl), alkoxy (especially lower alkoxy), lower alkoxyalkyl, alkoxyalkoxyalkyl, cycloalkyl, cycloalkylalkyl, phenylalkyl (especially benzyl), naphthylalkyl, phenoxyalkyl, alkenyl, and alkinyl, wherein the alkyl part of the substituent is unsubstituted or substituted by hydroxy, halogen, cyano or nitro, and the aromatic part of the substituent is unsubstituted or substituted by small substituents normally used in aromatic ring systems in agrochemicals such as lower alkyl, lower alkoxy, lower haloalkyl, lower haloalkoxy, lower alkylthio, cyano, nitro and halogen. Regulators based on the benzo-1,2,3-thiadiazole structure encompass all molecular systems capable of releasing the molecule actually acting as the regulator. A preferred group of regulators based on the benzo-1,2,3-thiadiazole structure includes benzo-1,2,3-thiadiazole-carboxylic acid, alkyl benzo-1,2,3-thiadiazolecarboxylate in which the alkyl group contains one to six carbon atoms, and substituted derivatives of these compounds. Suitable substituents include lower alkyl, lower alkoxy, lower alkylthio and halogen. In particular, benzo-1,2,3-thiadiazole-7-carboxylic acid and its alkyl esters, e.g. methyl benzo-1,2,3-thiadiazole-7-carboxylate, are preferred inducers for the chimeric DNA sequences comprising chemically regulatable DNA sequences isolated from PR protein genes. The syntheses of the mentioned chemical regulators and their utility as biocides may be discerned from British Patent 1,176,799 and Kirby, P. et al., J. Chem. Soc. C 2250 (1970). Among the preferred species based on the benzo-1,2,3-thiadiazole structure there may be mentioned, for example, benzo-1,2,3-thiadiazole-7-carboxylic acid, methyl benzo-1,2,3-thiadiazole-7-carboxylate, n-propyl benzo-1,2,3-thiadiazole-7-carboxylate, benzyl benzo-1,2,3-thiadiazole-7-carboxylate, benzo-1,2,3-thiadiazole-7-carboxylic acid secbutylhydrazide, and the like. An additional group of regulators for the chemically regulatable DNA sequences of this invention is based on the pyridine carboxylic acid structure, such as the isonicotinic acid structure and preferably the haloisonicotinic acid structure. Preferred are dichloroisonicotinic acids and derivatives thereof, for example the lower alkyl esters. Suitable regulators of this class of compounds are, for example, 2,6-dichloroisonicotinic acid, and the lower alkyl esters thereof, especially the methyl ester. The chemical regulators may be applied in pure form, in solution or suspension, as powders or dusts, or in other conventional formulations used agriculturally or in bioreactor processes. Such formulations may include solid or liquid carriers, that is, materials with which the regulator is combined to facilitate application to the plant, tissue, cell or tissue culture, or the like, or to improve storage, handling or transport properties. Examples of suitable carriers include silicates, clays, carbon, sulfur, resins, alcohols, ketones, aromatic hydrocarbons, and the like. If formulated as a conventional wettable powder or aqueous emulsion, the regulator formulation may include one or more conventional surfactants, either ionic or non-ionic, such as wetting, emulsifying or dispersing agents. The regulators may also be applied to plants in combination with another agent which is desired to afford some benefit to the plant, a benefit related or unrelated to the trait controlled by any chimeric gene which is regulated by the regulator. For example, a regulator can be admixed with a fertilizer and applied just before the expression of a transgenic trait unrelated to fertilization is desired. Or it can be combined with a herbicide and applied to mitigate the effect of the herbicide at the time when such effect would otherwise be at a maximum.

As a liquid formulation the regulator may be applied as a spray to plant leaves, stems or branches, to seeds before planting or to the soil or other growing medium supporting the plant. Regulators can also be used in bioreactor systems, regulation being achieved by a single addition of regulator formulation to the reaction medium or by gradual addition over a predetermined period of time. The regulator is applied in an amount and over a time sufficient to effect the desired regulation. A preferred regulator is one which shows no, or only minimal phytotoxic or other deleterious effect on the plant, plant tissue or plant cells to which it is applied in the amount applied.

A further aspect of the invention is a process for regulating transcription of a chemically inducible, but not wound or pathogen inducible gene, which process comprises applying such a chemical regulator to plant tissue, plant or seed containing a chemically regulatable nucleotide sequence as described supra. Preferred is such a process wherein the plant tissue, plant or seed contains a chemically regulatable nucleotide sequence mentioned above as being preferred.

It is another object of the present invention to provide recombinant nucleic acid molecules comprising a promoter according to the invention operably linked to a nucleotide sequence of interest. The nucleotide sequence of interest can, for example, code for a ribosomal RNA, an antisense RNA or any other type of RNA that is not translated into protein. In another preferred embodiment of the invention the nucleotide sequence of interest is translated into a protein product. The nucleotide sequence associated with the promoter sequence may be of homologous or heterologous origin with respect to the plant to be transformed. The sequence may also be entirely or partially synthetic. Regardless of the origin, the associated nucleotide sequence will be expressed in the transformed plant in accordance with the expression properties of the promoter to which it is linked. In case of homologous nucleotide sequences associated with the promoter sequence, the promoter according to the invention is useful for ectopic expression of said homologous sequences. Ectopic expression means that the nucleotide sequence associated with the promoter sequence is expressed in tissues and organs and/or at times where said sequence may not be expressed under control of its own promoter. In one particular embodiment of the invention, expression of nucleotide sequence associated with the promoter sequence suppresses its own expression and that of the original copy of the gene by a process called cosuppression. In a preferred embodiment of the invention the associated nucleotide sequence may code for a protein that is desired to be expressed in a chemically inducible, but not wound- or pathogen inducible fashion. Such nucleotide sequences preferably encode proteins conferring a desirable phenotypic trait to the plant transformed therewith. Examples are nucleotide sequences encoding proteins conferring antibiotic resistance, virus resistance, insect resistance, disease resistance, or resistance to other pests, herbicide tolerance, improved nutritional value, improved performance in an industrial process or altered reproductive capability. The associated nucleotide sequence may also be one that is transferred to plants for the production of commercially valuable enzymes or metabolites in the plant. Embraced by the present invention are also selectable or screenable marker genes, i.e. genes comprising a nucleotide sequence of the invention operably linked to a coding region encoding a selectable or screenable trait. Examples of selectable or screenable marker genes are described below. For certain target species, different antibiotic or herbicide selection markers may be preferred. Selection markers used routinely in transformation include the nptll gene which confers resistance to kanamycin, paromomycin, geneticin and related antibiotics (Vieira and Messing, 1982, Gene 19: 259-268; Bevan et al., 1983, Nature 304:184-187) the bacterial aadA gene (Goldschmidt-Clermont, 1991, Nucl. Acids Res. 19: 4083-4089), encoding aminoglycoside 3′-adenylyltransferase and conferring resistance to streptomycin or spectinomycin, the hph gene which confers resistance to the antibiotic hygromycin (Blochlinger and Diggelmann, 1984, Mol. Cell. Biol. 4: 2929-2931), and the dhfrgene, which confers resistance to methotrexate (Bourouis and Jarry, 1983, EMBO J. 2: 1099-1104). Other markers to be used include a phosphinothricin acetyltransferase gene, which confers resistance to the herbicide phosphinothricin (White et al., 1990, Nucl. Acids Res. 18: 1062; Spencer et al. 1990, Theor. Appl. Genet. 79: 625-631), a mutant EPSP synthase gene encoding glyphosate resistance (Hinchee et al., 1988, Bio/Technology 6: 915-922), a mutant acetolactate synthase (ALS) gene which confers imidazolione or sulfonylurea resistance (Lee et al., 1988, EMBO J. 7: 1241-1248), a mutant psbA gene conferring resistance to atrazine (Smeda et al., 1993, Plant Physiol. 103: 911-917), or a mutant protoporphyrinogen oxidase gene as described in EP 0 769 059. Selection markers resulting in positive selection, such as a phosphomannose isomerase gene, as described in patent application WO 93/05163, are also used. Identification of transformed cells may also be accomplished through expression of screenable marker genes such as genes coding for chloramphenicol acetyl transferase (CAT), β-glucuronidase (GUS), luciferase (LUC), and green fluorescent protein (GFP) or any other protein that confers a phenotypically distinct trait to the transformed cell. It is a further objective of the invention to provide recombinant expression vectors comprising a nucleotide sequence of the invention fused to an associated nucleotide sequence of interest. In these vectors, foreign nucleic acid molecules can be inserted into a polylinker region such that these exogenous sequences can be expressed in a suited host cell which may be, for example, of bacterial or plant origin. For example, the plasmid pBl101 derived from the Agrobacterium tumefaciens binary vector pBIN19 allows cloning and testing of promoters using beta-glucuronidase (GUS) expression signal (Jefferson et al, 1987, EMBO J 6: 3901-3907). The size of the vector is 12.2 kb. It has a low-copy RK2 origin of replication and confers kanamycine resistance in both bacteria and plants. There are numerous other expression vectors known to the person skilled in the art that can be used according to the invention. It is a further objective of the present invention to provide transgenic plants comprising the recombinant DNA sequences of the invention. The invention thus relates to plant cells, to plants derived from such cells, to plant material, to the progeny and to seeds derived from such plants, and to agricultural products with improved properties obtained by any one of the transformation methods described below. Plants transformed in accordance with the present invention may be monocots or dicots and include, but are not limited to, rice, maize, wheat, barley, rye, sweet potato, sweet corn, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar-beet, sunflower, rapeseed, clover, tobacco, carrot, cotton, alfalfa, potato, eggplant, cucumber, Arabidopsis thaliana, and woody plants such as coniferous and deciduous trees. Preferred plants to be transformed are rice, maize, wheat, barley, cabbage, cauliflower, pepper, squash, melon, soybean, tomato, sugar-beet, sunflower or cotton, but especially rice, maize, wheat, Sorghum bicolor, orchardgrass, sugar beet and soybean. The recombinant DNA sequences of the invention can be introduced into the plant cell by a number of well-known methods. Those skilled in the art will appreciate that the choice of such method might depend on the type of plant which is targeted for transformation, i.e., monocot or dicot. Suitable methods of transforming plant cells include microinjection (Crossway et al., 1986, Bio Techniques 4:320-334), electroporation (Riggs and Bates, 1986, Proc. Natl. Acad. Sci., USA 83:5602-5606), Agrobacterium-mediated transformation (Hinchee et al., 1988, Bio/Technology 6:915-922; EP 0 853 675), direct gene transfer (Paszkowski et al., 1984, EMBO J. 3:2717-2722), and ballistic particle acceleration using devices available from Agracetus, Inc., Madison, Wis. and Dupont, Inc., Wilmington, Del. (see, for example, U.S. Pat. No. 4,945,050 and McCabe et al., 1988, Bio/Technology 6:923-926). The cells to be transformed may be differentiated leaf cells, embryogenic cells, or any other type of cell. In the direct transformation of protoplasts, the uptake of exogenous genetic material into a protoplast may be enhanced by the use of a chemical agent or an electric field. The exogenous material may then be integrated into the nuclear genome. The previous work is conducted in dicot tobacco plants, which resulted in the foreign DNA being incorporated and transferred to progeny plants (Paszkowski et al., 1984, EMBO J. 3:2712-2722; Potrykus et al., 1985, Mol. Gen. Genet 199:169-177). Monocot protoplasts, for example, of Triticum monococcum, Lolium multiflorum (Italian rye grass), maize, and Black Mexican sweet corn, are transformed by this procedure. An additional preferred embodiment is the protoplast transformation method for maize as disclosed in EP 0 292 435, as well as in EP 0 846 771. For maize transformation also see Koziel et al., 1993, Bio/Technology 11:194-200. Transformation of rice can be carried out by direct gene transfer techniques utilizing protoplasts or particle bombardment. Protoplast-mediated transformation is described for Japonica-types and Indica-types (Zhang et al., 1988, Plant Cell Rep., 7:379-384; Shimamoto et al., 1989, Nature 338:274-276; Datta et al., 1990, Bio/Technology 8:736-740). Both types described above are also routinely transformable using particle bombardment (Christou et al., 1991, Bio/Technology 9:957-962). Patent application No. EP 0 332 581 describes techniques for the generation, transformation and regeneration of Pooideae protoplasts. These techniques allow the transformation of all Pooideae plants including Dactylis and wheat. Furthermore, wheat transformation is described in patent application No. EP 0 674 715; and by Weeks et al., 1993 (Plant Physiol. 102:1077-1084). The thus-constructed plant expression vector can, for example, be introduced into the calli of rice according to the conventional plant transformation method, and the differentiation of roots and leaves is induced therefrom, and then, can be transferred to a flowerpot for cultivation, thereby obtaining the transformed rice. The plants resulting from transformation with the DNA sequences or vectors of the present invention will express a nucleotide sequence of interest throughout the plant and in most tissues and organs. The genetic properties engineered into the transgenic plants described above are passed on by sexual reproduction or vegetative growth and can thus be maintained and propagated in progeny plants. Generally said maintenance and propagation make use of known agricultural methods developed to fit specific purposes such as tilling, sowing or harvesting. Specialized processes such as hydroponics or greenhouse technologies can also be applied. Use of the advantageous genetic properties of the transgenic plants according to the invention can further be made in plant breeding that aims at the development of plants with improved properties such as tolerance of pests, herbicides, or stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic plants according to the invention can be used for the breeding of improved plant lines that for example increase the effectiveness of conventional methods such as herbicide or pesticide treatment or allow to dispense with said methods due to their modified genetic properties. Alternatively new crops with improved stress tolerance can be obtained that, due to their optimized genetic “equipment”, yield harvested product of better quality than products that were not able to tolerate comparable adverse developmental conditions. It is another objective of the present invention to provide nucleotide sequences that can be used to express a nucleotide sequence of interest in a desired organism. Such molecules are commonly referred to as “promoters.” This organism can be a bacterium, a plant or any other organism of interest. Furthermore, the disclosure of SEQ ID NOs:1 to 3 enables a person skilled in the art to design oligonucleotides for polymerase chain reactions which attempt to amplify DNA fragments from templates comprising a sequence of nucleotides characterized by any continuous sequence of 15 and preferably 20 to 30 or more base pairs in SEQ ID NOs:1,2, or 3. Said nucleotides comprise a sequence of nucleotides which represents 15 and preferably 20 to 30 or more base pairs of SEQ ID NOs:1, 2, or 3. Polymerase chain reactions performed using at least one such oligonucleotide and their amplification products constitute another embodiment of the present invention.

### BRIEF DESCRIPTION OF THE SEQUENCES IN THE SEQUENCE LISTING

SEQ ID NO:1 part of the 5′ upstream sequence of the rice RCI-1 gene

SEQ ID NO:2 part of the rice RCI-1 gene including putative TATA box and putative start codon

SEQ ID NO:3 part of the rice RCI-1 gene including part of intron 1, exon 2, intron 2 and part of exon 3

SEQ ID NO:4 nucleotide sequence of the rice lipoxygenase RCI-1 transit peptide

SEQ ID NO:5 nucleotide sequence of the rice lipoxygenase RCI-1 cDNA

SEQ ID NO:6 amino acid sequence of the rice lipoxygenase RCI-1 transit peptide

SEQ ID NO:7 deduced amino acid sequence of the rice lipoxygenase RCI-1 cDNA

SEQ ID NO:8 degenerate primer

SEQ ID NO:9 anchored oligo dT reverse primer

SEQ ID NO:10 forward primer

SEQ ID NO:11 reverse primer

SEQ ID NO:12 reverse primer R1

SEQ ID NO:13 forward primer F1

SEQ ID NO:14 forward primer F2

SEQ ID NO: 15 primer attB1

SEQ ID NO:16 primer attB2

SEQ ID NO:17 nucleotide sequence of the about 4.5 kb Pstl/Pstl fragment from plasmid pBSK+LOX4a

SEQ ID NO:18 part of the 5′ upstream sequence of the rice RCI-1 gene obtained by PCR with forward primer F1 and reverse primer R1

SEQ ID NO:19 part of the 5′ upstream sequence of the rice RCI-1 gene obtained by PCR with forward primer F2 and reverse primer R1

SEQ ID NO:20 Gateway™ modified pNOV2347 binary Gateway™ destination vector with GIG reporter gene

SEQ ID NO:21 GIG, GUS intron GUS, GUS coding sequence with intron SEQ ID NO:22 pNOV6800 binary vector

Deposit

| Deposited material | Accession number | Date of deposit |
|---|---|---|
| pBSK + LOX4A | DSM 13524 | 06.06.2000 |
| pNOV6800 | NRRL B-30480 | May 25, 2001 |

The deposit of pBSK ⁺ LOX4A was made with the Deutsche Sammiung von Mikroorganismen und Zellkulturen GmbH (DSMZ), Mascheroder Weg 1b, D-38124 Braunschweig, Deutschland. The deposit of pNOV6800 was made with the Agricultural Research Service Culture Collection (NRRL), of the National Center for Agricultural Utilization Research, Agricultural Research Service, United States Department of Agriculture, 1815 North University Street, Peoria, Ill. 61604 USA.

Below are illustrative examples of the present invention. The present invention is not to be limited in scope by the specific embodiments described which are intended as single illustrations of individual aspects of the invention, and any constructs, promoters, transit peptides or enzymes which are functionally equivalent are within the scope of this invention.

### EXAMPLES

Standard recombinant DNA and molecular cloning techniques used here are well known in the art and are described, for example, by Sambrook et al., 1989, “Molecular Cloning”, Cold Spring Harbor, Cold Spring Harbor Laboratory Press, NY and by Ausubel et al.,1994, “Current protocols in molecular biology”, John Wiley and Sons, New York.

### Example 1: Plant Material and Treatment

Rice plants (Otyza sativa cv. Kusabue) are grown in pots with clay soil that are soaked with an iron fertilizer solution (Gesal Pflanzen Tonic, Novartis, Basel, Switzerland) under a 16 h light/8 h dark cycle at 25° C. and 80% humidity. M. glisea (race 007 and 031 from the Institute of Biochemistry, Faculty of Agriculture, Tamagawa University, Machida-shi, Tokyo 194, Japan) is cultivated on oat-meal starch agar (30 g l⁻¹ oat-flakes, 20 g l⁻¹ agar-agar, 10 g l⁻¹ starch and 2 g l⁻¹ yeast extract). After incubation at 27° C. for 2 weeks, aerial mycelia are removed with a sterile spatula and synchronous sporulation is induced by further incubation under black light (310-360 nm). For inoculations the concentration of conidia is adjusted to 1×10⁶ ml⁻¹ in a spraying solution (1 g l⁻¹ gelatine, 0.1% Tween-20). Plants are inoculated 12 - 14 days after sowing by spraying the conidial suspension onto the leaves. After an incubation for 24 h in a dark moist chamber (26° C., approx. 100% relative humidity), plants are kept in a humid atmosphere under the same temperature and light regime as described above. Plant treatment with the chemical inducers is done 10 days after sowing at the emergence of leaf 3. All chemical concentrations are given as ppm (mg active ingredient 1 of applied solution). Probenazole is applied as a 250 ppm solution of the pure substance by soil drench as described (Thieron et al. (1995) Systemic acquired resistance in rice: Studies on the mode of action of diverse substances inducing resistance in rice to Pyricularia oryzae. Mededelingen Faculteit Landbouwkundige en Toegepaste Biologische Wetenschappen Universiteit Gent 60, 421-430). Formulations of BTH (1:1 (w/w) mixture of active ingredient and wettable powder) and INA (1:3 (w/w) mixture of active ingredient and wettable granulate) are applied onto leaves by spraying. All controls are done by application of spray-solutions without active substance. Jasmonic acid is applied as a 1 mM solution in ethanol as described (Schweizer et al. (1997) Plant Physiol. 114, 79-88). Wounding and measurement of gene expression in systemic tissue is done according to (Schweizer et al. (1998) Plant J. 14, 475-481).

### Example 2: cDNA Library Construction

Total RNA is extracted from rice leaves treated with 100 ppm INA and harvested after 24 and 48 hours. PolyA+-RNA is prepared as described in Example 2 and equal amounts from both time points are pooled. A cDNA library is constructed using the lambda Zap Express cDNA Synthesis Kit (Stratagene, La Jolla, Calif.) according to the manufacturer's instructions.

### Example 3: Cloning of the Rice Lipoxygenase cDNA RCI-1

A. Cloning of a Rice Lipoxygenase cDNA Fragment by PCR

A PCR-based strategy is used to generate a lipoxygenase cDNA fragment from INA-treated rice leaves. By aligning two lipoxygenase sequences from rice (Peng et al. (1994) J. Biol. Chem. 269, 3755-3761; Ohta et al. (1992) Eur. J. Biochem. 206, 331 -336) and one from wheat (Görlach et al. (1996) Plant Cell. 8, 629-643), several conserved regions are identified, one of which is near the C-terminus and contains the amino acid sequence HAAVNFG that is invariant in all three sequences. Total RNA is extracted as described (Dudler & Hertig (1992) J. Biol. Chem. 267, 5882-5888) from untreated control leaves and from leaves sprayed with a 100 ppm INA solution 24 and 48 hours after treatment. Poly A⁺-RNA is prepared from total RNA using the quick mRNA isolation kit from Stratagene (La Jolla, Calif.). The polyA⁺-mRNA samples from the two time points are pooled and 1 μg aliquots of poly A⁺ RNA are used as templates for RT-PCR using the degenerate oligonucleotide 5′-CAYGCNGTNAANTTYGG-3′ (SEQ ID NO:8), which corresponds to the HAAVNFG amino acid sequence motif in the C-terminal region of the rice RLL2 lipoxygenase (Peng et al. (1994) J. Biol. Chem. 269, 3755-3761) as the forward primer and an anchored oligo-dT reverse primer (5′-AATGCTTTTTTTTTTTTTTTV-3′, SEQ ID NO:9).

Reverse transcription is done as follows:

1 μl RNA (1 μg/ml)

8 μl 5×RT-buffer

4 μl anchored oligo-dT reverse primer (100 pmol/μl)

4 μl dNTP (10 mM each)

18 μl H₂O_{DEPC}

are mixed and incubated at 94° C. for 15 min. This is followed by 1 h incubation at 42° C. Five minutes after the transfer to 42° C., 3 μl AMV-RT (Boehringer)and 2 μl RNAse Inhibitor (Boehringer) are added. Reverse transcription is terminated by 5 min incubation at 75° C. PCR with reverse transcribed RNA is done as follows:

3 μl cDNA (see above)

10 μl 10×PCR-buffer

1 μl anchored oligo-dT reverse primer (100 pmol/μl)

1 μl degenerate primer (100 pmol/μl)

2 μl dNTP (10 mM each)

0,5 μl Taq DNA polymerase (Boehringer)

82,5 μl H₂O

are mixed and subjected to the following program:

| | | | |
|---|---|---|---|
| 1 cycle | 94° C./15 min | 39° C./2 min. | 72° C./3 min |
| 35 cycle | 94° C./30 sec. | 39° C./30 sec. | 72° C./1 min |
| 1 cycle | 94° C./1 min | 39° C./2 min. | 72° C./10 min |

Then, 0,5 μl fresh Taq DNA polymerase are added, and 20 more cycles are performed under the conditions given above.

PCR products derived from treated and untreated leaves are visualized on ethidiumbromide-stained agarose gels. A PCR product of approximately 600 bp arises only in the INA-treated sample but not in the control. The piece of gel corresponding to the about 600 bp band present only in the lane with INA-treated probes is cut out. The DNA is subsequently eluted from the gel and cloned into the pGEMT_{easy} vector (Promega, Madison, USA) and the resultant plasmid designated pKL-5.

B. cDNA library screening to obtain a full-length rice lipoxygenase cDNA clone The ³²P-labelled insert of pKL-5 is used as a probe to screen a lambda cDNA library constructed from INA-treated rice leaves (see Example 2). Positive clones are purified. The one with the largest insert is designated RCI-1 rice chemically induced cDNA 1) and subcloned into the pBK-CMV (Stratagene) phagemid vector by in vivo excision according to the instructions of the manufacturer. The resulting plasmid is called pRCI-1. The RCI-1 insert is sequenced on both strands by primer walking using CY5-labelled primers and an ALF DNA-sequencer (Pharmacia, Uppsala, Sweden). The RCI-1 cDNA insert (SEQ ID NO:5) consists of 3018 bp and contains an open reading frame of 2766 bp (from base 48 to base 2816 of SEQ ID NO:5) encoding a protein of 922 amino acid residues (SEQ ID NO:7) with a predicted Mr of 105 kDa. The presumed translation initiation site is the first methionine codon in the open reading frame. Sequence comparison revealed that the RCI-1 protein was most similar to the barley LOX2:Hv:1 (Vörös et al. (1998) Eur. J. Biochem. 251, 36-44), showing 60% identity and 68% similarity at the amino acid level. Sequence similarity (identity) to the two already published rice lipoxygenase forms at the amino acid level were 52% (43%) in comparison to L-2 (Ohta et al. (1992) Eur. J. Biochem. 206, 331-336) and 58% (50%) to RLL2 (Peng et al. (1994) J. Biol. Chem. 269, 3755-3761), respectively. The RCI-1 rice lipoxygenase has an N-terminal extension (SEQ ID NO:6, corresponding to amino acid 1 to 36 of SEQ ID NO:7) that is thought to direct this class of proteins to plastids, in particular to chloroplasts. This putative chloroplast targeting sequence clearly separates this class of lipoxygenase (LOX) species from another class that is predominately found in kernels and seedlings and that includes LoxA, LoxB, and LoxC from barley (van Mechelen et al. (1999) Plant Mol. Biol. 39, 1283-1298), and LOX L-2 from rice (Ohta et al. (1992) Eur. J. Biochem. 206, 331-336).

### Example 4: Southern Blot Analysis

Genomic DNA is extracted from rice leaves using a CTAB procedure (Ausubel et al. (1987) Current protocols in molecular biology, Wiley and Sons, New York). Digestion with restriction enzymes, electrophoretic separation on agarose gels, and transfer to GeneScreen membranes (Dupont NEN, Brussels, Belgium) are performed according to standard procedures. Filters are hybridized to a ³²P-labeled probe consisting of an EcoRI/HindIII fragment of pRCI-1 that contains the first 1280 bp of the RCI-1 cDNA in 1 M NaCl,1% SDS, 10% dextrane sulphate, and 100 μg ml⁻¹ denatured salmon sperm DNA overnight at 68° C. Filters are washed in 0.2×SSC (1×SSC is 150 mM NaCl; 15 mM sodium citrate); 0.1% SDS at 65° C. When DNA digested with a number of enzymes that do not cut within the sequence of the probe is analyzed, one to three bands hybridizing to the probe are detected. This suggests that in addition to RCI-1, there is at least one other rice gene that weakly crosshybridizes with the probe.

### Example 5: RCI-1 Expression Studies

The effect of various stimuli on the abundance of RCI-1 transcripts is investigated using RNA gel blot analysis. For this, total RNA is extracted from treated and untreated leaves as described (Dudler. & Hertig. (1992) J. Biol. Chem. 267, 5882-5888). For gel blot analysis, 10 μg of total RNA is loaded per slot and separated on formaldehyde agarose gels, transferred to GeneScreen membranes, and cross-linked using an UV crosslinker (Amersham, UK). Loading of the lanes is monitored by ethidium bromide staining of the gel before transfer. Filters are hybridized to a ³²P-labeled probe consisting of an EcoRI/HindIII fragment of pRCI-1 that contains the first 1280 bp of the RCI-1 cDNA in 1 M NaCl, 1% SDS, 10% dextrane sulphate, and 100 μg ml⁻¹ denatured salmon sperm DNA overnight at 68° C.. Filters are washed in 0.2×SSC (1×SSC is 150 mM NaCl; 15 mM sodium citrate); 0.1% SDS at 65° C.. A 528 bp cDNA fragment encoding part of the rice ribosomal Protein L3 (RP-L3, accession number D12630) is used as a probe for a constitutively expressed transcript. This fragment is fortuitously amplified and cloned together with the partial lipoxygenase clone pKL-5. A time course experiment with rice leaves that have been treated with INA is analyzed by RNA gel blot analysis. The hybridization signal appears as a distinct band corresponding to an RNA of approximately 3200 bp length and a smeared signal of about 1200 to 1700 bp. By stripping and reprobing the same membrane with a constitutively expressed gene (RP-L3), the high quality of the RNA preparation is confirmed. The smeared signal thus indicates that RCI-1 transcripts are particularly unstable, perhaps due to a high turn-over rate. The time course experiment reveals that RCI-1 transcripts starts to accumulate 8 hours after treatment and reaches maximal levels after 24 to 48 h. Similarly, treatment with the resistance activator BTH, a functional analogue of INA, also induces RCI1 transcript accumulation as does the application of probenazole (tradename oryzemate), which represents a different class of resistance-inducing chemicals (Kessmann et al (1994) Ann. Rev. Phytopathol. 32, 439-459). The delayed time course of RCI-1 mRNA accumulation in response to probenazole treatment may rather reflect the different mode of application, i. e. spraying onto the leaves in case of INA and BTH vs. soil drenching with probenazole, respectively, than a difference in signaling. Thus, RCI-1 transcripts accumulate upon application of a number of different chemical resistance inducers. In contrast, RCI-1 mRNA levels neither increase after inoculation with the non-host pathogen P. syringae pv. syringae, a biological inducer of resistance against rice blast (Smith & Métraux (1991) Physiol. Mol. Plant Pathol. 39, 451-461), nor upon infection with M. grisea, the causal agent of rice blast. Furthermore, RCI-1 transcript levels strongly increase 7 to 12 h after spraying of a 1 mM jasmonic acid (JA) solution onto rice leaves. Interestingly, wounding, which is known to increase endogenous levels of JA in rice and induces increased systemic protection against blast infection (Schweizer et al. (1998) Plant J. 14, 475-481; Schweizer et al. (1997) Plant Physiol. 114, 79-88), does not activate RCI-1 transcription, neither locally nor systemically. To investigate whether the observed accumulation of RCI-1 transcripts after treatment with chemical inducers correlates with the increase in lipoxygenase (LOX) enzyme activity in rice leaves. To this end LOX activity is measured in the BTH-treated rice leaves that are also used for RNA gel blot analyses shown (see above). Consistent with the results of the RNA gel blot analysis, a significant increase in enzymatic activity is observed between 24 and 48 h after BTH treatment. In addition, the BTH dose that is sufficient to trigger RCI-1 transcript accumulation also causes an increase in LOX enzyme activity. Both results are compatible with the assumption that the increase in enzyme activity is predominantly due to the activation of the RCI-1 (and homologous) gene(s). To analyze this hypothesis further, RNA derived from BTH-treated rice plants is probed with other rice LOX cDNAs that correspond to a pathogen-induced gene (RLL2; Peng et al. (1994) J. Biol. Chem. 269, 3755-3761) and a gene expressed in seedlings (L-2, Ohta et al. (1992) Eur. J. Biochem. 206, 331-336). In contrast to RCI-1 mRNA, RLL2 and L-2 transcripts do not accumulate after treatment with INA, BTH, and probenazole.

### Example 6: Expression of RCI-1 in E. coli

The RCI-1 coding region is placed under the control of an IPTG-inducible promoter of an E. coli expression vector. More specifically, the RCI-1 cDNA is cloned into the pDS56/RBSII, Sphl expression vector (Stüber et al. (1990) System for high-level production in Escherichia coli and rapid purification of recombinant proteins: Applications to epitope mapping, preparation of antibodies, and structure-function analysis. In Immunological Methods (Levkovits, I. & Pernis, B., eds) pp. 121-152. Academic Press, New York) from which the unique Pstl site is eliminated by Pstl digestion and religation after blunting the sticky ends using T4 DNA polymerase. The new vector is named pDS56/RBSII, Sphl (−Pstl). An Sphl site is introduced at the translation initiation site of the RCI-1 cDNA by PCR amplification of a 146 bp RCI-1 fragment using the forward primer 5′-GTCAGCATGCTCACGGCCAC-3′ (SEQ ID NO:10; the Sphl site is underlined; the translation initiation codon is given in italics) and the reverse primer 5′- CATTGACGACCTCCGACAAG-3′ (SEQ ID NO:11), which anneals downstream of an internal Xhol site (nucleotide position 149 of SEQ ID NO:5). The amplified fragment is cut with Sphl and Xhol and ligated together with the 2.3 kb Xhol/BamHI fragment containing the middle part of the RCI-1 cDNA (nucleotide position 149 to 2468 of (SEQ ID NO:5) in a single reaction into pDS56/RBSII, Sphl (−Pstl) that has been digested with Sphl and BamHI. The resulting vector (pExpr1) is checked by restriction analysis. pExpr1 is then cut with Pstl (at position 891 in the top strand of the cDNA insert, corresponding to base 891 of SEQ ID NO:5) and SalI (in the multiple cloning site of pDS56/RBSII, Sphl (−Pstl) downstream of the insert), and the resulting cDNA fragment is replaced with the corresponding Pstl/Xhol fragment of pRCI-1 (Xhol cleaves in the multiple cloning site downstream of the cDNA insert). The resulting construct (pExprRCI-1), which contains the complete RCI-1 coding region under the control of an IPTG-inducible promoter, is subsequently transformed into M15 E. coli cells (Stüber et al. (1990) System for high-level production in Escherichia coli and rapid purification of recombinant proteins: Applications to epitope mapping, preparation of antibodies, and structure-function analysis. In Immunological Methods (Levkovits, I. & Pernis, B., eds) pp. 121-152. Academic Press, New York). Production of recombinant RCI-1 protein is induced by addition of 1 mM IPTG (final concentration) to a 250 ml E. coli M15 culture grown to an OD₆₀₀ of 1 and further incubation on a shaker overnight at 19° C. The cells are harvested by centrifugation (5000 g, 10 minutes) and resuspended in 5 ml lysis buffer (50 mM Na-phosphate buffer pH 7.5 containing 1 mg l-1 lysozyme). After a 30 minute incubation on ice, the lysate is centrifuged (12000 g, 15 minutes) and the pellet is transferred into a mortar and ground in extraction buffer (0.1 M K-phosphate buffer pH 7, 30 mg polyvinyl-poly-pyrrolidone, 1 mM EDTA). After centrifugation, the clear supernatant is used as an enzyme preparation for further biochemical analysis. SDS-PAGE analysis of extracts of E. coli transformed with this construct reveals a novel protein with a molecular mass of about 103 kDa which is recognized by a LOX specific antibody on a western-blot. This size is compatible with the predicted value of 105 kDa (see Example 3).

### Example 7: Biochemical Analysis of RCI-1 Lipoxygenase Activity

Lipoxygenase activity is measured at 30° C. photospectrometrically at 234 nm using linoleic acid as a substrate and 5-20 μl from the recombinant RCI-1 enzyme extract (Bohland et al. (1997) Plant Physiol. 114, 679-685). For determination of the pH optimum, different buffers with overlapping pH ranges (pH 4-6: 0.1 M Na-acetate; pH 6-8: 0.1 M Na-phosphate; pH 8-10: 0.1 M Tris-HCl) are used. The molar extinction coefficient of the reaction product, 2.5×10⁷ cm⁻¹ mol⁻¹, is used for the calculation of the enzyme activity. Crude extracts of these bacteria exhibit increased LOX activity using linoleic acid as a substrate, while control extracts of E. coli without expression construct or containing the empty vector do not have detectable LOX activity. Maximal activity is observed around pH 8 to 9, indicating that RCI-1 must be classified as a type 1 LOX (Siedow (1991) Ann. Rev. Plant Physiol. Plant Mol. Bio. 42, 145-188). However, it should be noted that recently a second classification based on the presence of a plastomic transit peptide is introduced (Shibata et al. (1994) Plant Mol. Biol. Rep. 12, 41-42). According to this scheme, RCI-1 must be classified as a type 2 LOX. The products of the enzymatic activity of the RCI-1 protein are analyzed by HPLC (Bohland et al. (1997) Plant Physiol. 114, 679-685). Approximately 0.2 nkat enzyme activity obtained from recombinant RCI-1 protein is incubated with 50 μl substrate solution (10 μl of linoleic or α-linolenic acid, respectively; 20 μl ethanol, 20 μl H₂O) in 2 ml 0.1 M Tris-HCl pH 8.8 for 20 min. at 30° C. The reaction is stopped by lowering the pH to 3.0 with diluted HCl, and the hydroperoxides are extracted with 1 ml of CHCl₃ followed by two washes with water. The reaction products are subjected to HPLC-analysis (4-μm particle size, Suprasphere-Si, 4.6×125 mm; Merck, Darmstadt, Germany). Isocratic elution is performed with hexane:2propanol:acetonitrile:acetic acid (98.3:1.5:0.1:0.1, v/v/v/v) at a flow rate of 1 ml min⁻¹. Products are detected at 234 nm and standards are obtained from Biomol (Hamburg, Germany) or prepared from linoleic or α-linolenic acid by incubation with soybean lipoxygenase, respectively, as described (Bohland et al. (1997) Plant Physiol. 114, 679-685). When the reaction products of recombinant RCI-1 are analyzed by HPLC, (13S)-hydroperoxy-(9Z,11 E, 15Z)-octadecatrienoic acid (13-HPOD) is the predominant product, irrespective of whether linoleic acid or linolenic acid served as a substrate for the enzyme. (9S)-hydroperoxy-(10E, 12Z, 15Z) octadecatrienoic acid (9-HPOD) is only detected in minor amounts.

### Example 8: RCI-1 Transit Peptide::GFP Reporter-Gene Construction and Transformation

A chimeric gene is constructed that encodes a fusion protein containing the N-terminal 37 amino acids of the RCI-1 protein (SEQ ID NO:6) followed by four amino acids resulting from the cloning procedure followed by GFP sequence. To this end, pRCI-1 (see Example 3 B) is digested with Xhol, which cuts the top strand after position 149 of the cDNA insert (corresponds to base 149 of SEQ ID NO:5) and in the multiple cloning site downstream of the insert, and religated. The resulting plasmid contains the first 158 bp of the RCI-1 cDNA, since the nucleotide sequence of the vector downstream of the cloning site is identical to base 150 to base 158 of SEQ ID NO:5. This plasmid is referred to as pRCI158. Its insert comprising the transit peptide cDNA (SEQ ID NO:4) is cleaved out with EcoRI and Xbal, which both cut in the multiple cloning site, and the sticky ends are blunted by filling them in with Klenow enzyme. The blunted fragment is cloned into the filled-in and dephosphorylated Spel cloning site of the binary pCambia 1302 vector (Cambia, Canberra, Australia), which contains the mGFP-5 coding region (MRC Laboratory of Molecular Biology, Cambridge, England). The correct orientation of the inserted fragment is checked by restriction digestion and the final construct is verified by sequencing. The construct is designated pRCI transit peptide::GFP and transformed into the Agrobacterium tumefaciens strain LBA 4404 by triparental mating. Transformation of Arabidopsis leaf cells is achieved by infiltration of Agrobacterium into intact leaves of Arabidopsis thaliana, ecotype Wassiljewskija (Ws), 14 days after germination according to Kapila et al. (1997) (Plant Sci. 122, 101-108). Expression of the fusion protein is monitored 2 days after transformation by confocal imaging using a Leica-TCS confocal laser scanning microscope and a PLAPO×100 oil immersion objective (Leica Microsystems, Heidelberg, Germany) with the following filter settings: excitation 476/488 nm; GFP-emission 515-552 nm, chlorophyll-emission 673-695 nm. GFP fluorescence and chlorophyll autofluorescence are recorded simultaneously using independent 2-channel-detection. Confocal imaging of leaves from transgenic Arabidopsis plants expressing the pRCI transit peptide::GFP construct reveals a strict congruence of GFP fluorescence and chlorophyll autofluorescence, indicating that the fusion protein is localized in the chloroplasts.

### Example 9: Cloning of the RCI-1 Promoter Region

A. Screening of a λ-DASH Genomic DNA Library of Rice

A λ-DASH II/BamHI DNA library representing genomic DNA derived from Oryza sativa cv. Norin plants is constructed according to the protocol of Stratagene (La Jolla, USA). The titer of the library is determined to be 2.12×10¹⁰ pfu/ml. Screening of the library is carried out following the protocol of Stratagene. The library is plated on four 530 cm² bio-assay dishes (Nalge Nunc Int., Naperville, USA) containing NZY agar. The density is adjusted to 150'000 pfu/plate and plating is carried out with E. coli XL1 -blue MRA (Stratagene, La Jolla, USA) as a host strain according to the protocol of Stratagene. The plaques are transferred onto a nylon membrane (Hybond™ 0.45 μm, Amersham, Uppsala, Sweden) and the DNA is crosslinked in a UV crosslinker (Amersham, Uppsala, Sweden). A 900 bp Pstl-fragment representing the 5′-prime end of the rice RCI-1 lipoxygenase cDNA clone pRCI-1 (SEQ ID NO:5) is labeled with ³²p and hybridized overnight at 65° C. to the plaque lifts according to standard procedures (Maniatis et al., 1982). Two additional rounds of screening resulted in a positive λ-clone (λLOX4).

B. Subcloning of the Putative LOX Promoter Region

Liquid lysate DNA preparations of the λ-clone are prepared according to standard procedures and analyzed by digestion with the Pstl restriction enzyme and gel electrophoresis on a 0.6% (w/v) agarose gel. Southern blotting and subsequent hybridization of the membrane to the 900 bp Pstl-fragment of the RCI-1 cDNA are done according to standard procedures. A strong band corresponding to a 4.5 kb fragment of λLOX4 is detected. The 4.5 kb DNA fragment is subcloned into a pBluescript/SK⁺ vector (Stratagene, La Jolla, USA). Transformation of E. coli strain DH5a cells is done according to standard procedures and transformants are selected on LB Agar containing Ampicillin (100 μg/ml). This resulting clone is designated pBSK+LOX4A. Clone pBSK+LOX4A is deposited with the DSMZ (Deutsche Sammiung von Mikroorganismen und Zellkulturen GmbH on Jun. 6, 2000 with accession number DSM 13524. Clone pBSK+LOX4A contains the RCI-1 lipoxygenase promoter on a 4.5 kb Pstl/Pstl fragment and is further analyzed by DNA sequencing. Clone pBSK+LOX4A comprises, in a 5′ to 3′ direction, the nucleotide sequences depicted in SEQ ID NOs:1, 2 and 3. SEQ ID NO:1 comprises the 5′-end of the 4.5 kb Pstl/Pstl fragment. This nucleotide sequence is 358 nucleotides in length and contains at its 5′ end in position 1 to 6 the Pstl-site. The region between SEQ ID NO:1 and SEQ ID NO:2 of the 4.5 kb Pstl/Pstl fragment is between about 240 and 440 bp in length. The central region of the 4.5 kb Pstl/Pstl fragment is shown in SEQ ID NO:2 and is 2104 bp in length. It contains the putative TATA box (position 1261 to 1266 SEQ ID NO:2), the putative start codon (position 1359 to 1361 of SEQ ID NO:2), as well as the 5′ untranslated region and nucleotide sequences upstream of the putative TATA box. Comparison of the genomic DNA (SEQ ID NO:2) and the cDNA (SEQ ID NO:5) shows that the sequences located at position 1312 to 1701 of SEQ ID NO:2 comprise all or part of exon 1, and the sequences located at position 1702 to 2104 of SEQ ID NO:2 are the 5′ part of intron 1. The region between SEQ ID NO:2 and SEQ ID NO:3 of the 4.5 kb Pstl/Pstl fragment is between about 85 and 285 bp in length. The 3′ end of the 4.5 kb Pstl/Pstl fragment is shown in SEQ ID NO:3. This sequence depicts a nucleotide sequence of 1516 bp in length. It contains, in a 5′ to 3′ direction, the 3′ end of intron 1 (position 1 to 97 of SEQ ID NO:3) followed by exon 2 (position 98 to 366 of SEQ ID NO:3), intron 2 (position 367 to 1283 of SEQ ID NO:3) and part of exon 3 (position 1284 to 1516 of SEQ ID NO:3). The Pstl site is located at position 1511 to 1516.

### Example 10: Plasmid Amplification and DNA Sequencing of the Clone pBSK+LOX4A

Transformants are grown at 37° C. in a 50 ml over-night culture of LB Medium containing Ampicillin (100 μg/ml). Cells are harvested and plasmid DNA is extracted using the Jetstar Midi plasmid extraction kit (Genomed GmbH, Bad Oeynhausen, Germany). Sequencing of the clone pBSK+LOX4A is carried out by the chain termination method (Maniatis et al. (1982) Molecular Cloning. A Laboratory Manual. Cold Spring Harbor Laboratory Press, Cold Spring Harbor). Sequencing reactions are performed the BigDye™ terminator cycle sequencing kit (Perkin-Elmer Corp., Norwalk, Conn.) according to the instructions of the manufacturer and the sequences are determined with a 373 DNA-sequencer (Applied Biosystems, Foster City, Calif.). They are assembled and analyzed using the Wisconsin Sequence Analysis package (Genetics Computer Group, Madison, Wis.). Ambiguities are clarified by comparison with the corresponding electropherogram print. SEQ ID NO:1 corresponds to the 5′ end, SEQ ID NO:2 to the middle and SEQ ID NO:3 to the 3′ end of the 4.5 kb insert of pBSK+LOX4A.

### Example 11: Preparation of a CaMV 35S Promoter::RCI-1 cDNA Construct

The starting plasmid is the plant binary vector pBl 121 (Clontech, Palo Alto, Calif.), which contains the GUS reporter gene under the control of the CaMV 35S promoter. The GUS reporter gene is removed from pBI 121 and replaced with the RCI-1 cDNA. To this end, pBI 121 is digested with the restriction enzyme Sst I, and the sticky ends are filled in with dNTPs and T4 DNA polymerase according to standard procedures. After cutting with Sma I, the vector fragment is separated from the GUS reporter gene by agarose gel electrophoresis and religated. This vector is named pBl 121 (-GUS). pBl 121 (-GUS) is cut with Bam Hi, the sticky ends are blunted by filling them in with dNTPs and T4 DNA polymerase, and the RCI-1 cDNA fragment is ligated into this vector, after it has been cut out of pRCI-1 with Eco RI and Xba I and its sticky ends have been blunted with dNTPs and T4 DNA polymerase. After transformation into E. coli, plasmid is prepared from a number of colonies. The orientation of the RCI-1 fragment is checked by restriction digestion using Xba I, which cuts immediately upstream of the insert, and Bam HI, which cuts the top strand of the RCI-1 cDNA after nucleotide 2688. The correct orientation results in a fragment of approx. 2700 bp in length, the wrong orientation in a fragment of approx. 350 bp. Then a plasmid which contains the RCI-1 cDNA in the correct orientation, i. e. such that the filled in Eco RI site is next to the CaMV 35S promoter, is selected and designated p35Spromoter::RCI-1 cDNA. For Agrobacterium mediated transformation, the plasmid is transformed into the Agrobacterium tumefaciens strain LBA 4404 by electroporation.

### Example 12: Transformation of Rice

The transformed Agrobacterium tumefaciens strain is grown for 3 days in the AB liquid medium supplemented with 30 mg/L hygromycin B and 3 mg/L tetracycline, and it is cocultivated with three-week-old calli which are induced from the scutellum of mature seeds in the N6 medium (Chu, C.C. et al., Sci, Sin., 18, 659-668(1975)) containing 2 mg/L 2,5-D, on the 2N6-As medium supplemented with 1 mM betaine (Hiei, Y. et al., Plant J., 6, 271-282(1994)) in darkness at 25° C. for 2-3 days. The co-cultivated calli are washed with sterile water containing 100 mg/L cefotaxime, and again incubated on an N6 medium containing 40 mg/L hygromycin and 250 mg/L cefotaxime for 3 weeks. Actively growing hygromycin-resistant calli are transferred onto the selection medium [for example, MS media (Life Technologies) +0.2 mg/L NAA (naphthalene acetic acid)+2 mg/L kinetin+2% sorbitol+1.6% phytagar (Gibco)+50 mg/L hygromycin B+250 mg/L cefotaxime], and then cultivated for 2-3 weeks under continuous light condition of 40 pmol m⁻² s⁻¹. The thus-obtained plantlets are potted and grown in a growth chamber under 10 h light/14 h dark condition to obtain transgenic rice plants.

### Example 13: Transformation of Maize

Type I embryogenic maize callus cultures (Green et al, Miami Winter Symposium 20,1983) are initiated from immature embryos, 1.5-2.5 mm in length, from greenhouse grown material. Embryos are aseptically excised from surface-sterilized ears approximately 14 days after pollination. Embryos may be placed on D callus initiation media with 2% sucrose and 5 mg/L chloramben (Duncan et al, Planta 165: 322-332,1985) or onto KM callus initiation media with 3% sucrose and 0.75 mg/L 2,4-d (Kao and Michayluk, Planta 126:105-110,1975). Embryos and embryogenic cultures are subsequently cultured in the dark. Embryogenic responses are removed from the explants after ˜14 days. Embryogenic responses from D callus initiation media are placed onto D callus maintenance media with 2% sucrose and 0.5 mg/L 2,4-d while those from KM callus initiation media are placed onto KM callus maintenance media with 2% sucrose and 5 mg/L Dicamba. After 3 to 8 weeks of weekly selective subculture to fresh maintenance media, high quality compact embryogenic cultures are established. Actively growing embryogenic callus pieces are selected as target tissue for gene delivery. The callus pieces are plated onto target plates containing maintenance medium with 12% sucrose approximately 4 hours prior to gene delivery. The callus pieces are arranged in circles, with radii of 8 and 10 mm from the center of the target plate. Plasmid DNA containing the promoter-RCI-1 cDNA construct or a RCI-1-promoter-reporter gene construct is precipitated onto gold microcarriers as described in the DuPont Biolistics manual. Two to three μg of each plasmid is used in each 6 shot microcarrier preparation. Genes are delivered to the target tissue cells using the PDS-1000 He Biolistics device. The settings on the Biolistics device are as follows: 8 mm between the rupture disc and the macrocarrier, 10 mm between the macrocarrier and the stopping screen and 7 cm between the stopping screen and the target. Each target plate is shot twice using 650 psi rupture discs. A 200×200 stainless steel mesh (McMaster-Carr, New Brunswick, N.J.) is placed between the stopping screen and the target tissue. Seven days after gene delivery, target tissue pieces are transferred from the high osmotic medium to selection media. For selection using the BAR gene, target tissue pieces are placed onto maintenance medium containing 100 mg/L glufosinate ammonium (Basta®) or 20 mg/L bialaphos (Herbiace®). All amino acids are removed from the selection media. After 5 to 8 weeks on these high level selection media, any growing callus is subcultured to media containing 3-20 mg/L Basta®. For selection using the Mannose Phosphate Isomerase gene, target tissues are placed on their respective maintenance media containing no sucrose and 1% mannose. The amino acids are not removed from these media. After 5 to 8 weeks, growing callus is either subcultured to D callus maintenance medium containing no sucrose and 1.5% mannose or KM callus maintenance medium containing 1% sucrose and 0.5% mannose. Embryogenic callus growing on selection media is subcultured every 2 weeks for 4 to 8 weeks until enough callus is produced to generate 10-20 plants. Tissue surviving selection from an original target tissue piece is subcultured as a single colony and designated as an independent transformation event. At that point, colonies selected on Basta® are transferred to a modified MS medium (Murashige and Skoog, Physiol. Plant, 15:473-497, 1962) containing 3% sucrose (MSS) with no selection agent and placed in the light. Either 0.25 mg/L ancymidol and 0.5 mg/L kinetin are added to this medium to induce embryo germination or 2 mg/L benzyl adenine is added. Colonies selected using mannose are transferred onto a modified MS medium containing 2% sucrose and 1% mannose (MS2S+1M) with the ancymidol and kinetin additions described above or a modified MS medium containing 2% sucrose and 0.5% mannose (MS2S+0.5M) with the benzyl adenine addition described above. Regenerating colonies from Basta® selection are transferred to MS3S media without ancymidol and kinetin or benzyl adenine after 2 weeks. Regenerating colonies from mannose selection are transferred to MS2S+1M and MS2S+0.5M media respectively without hormones after 2 weeks. Regenerating shoots with or without roots from all colonies are transferred to Magenta boxes containing MS3S medium and small plants with roots are eventually recovered and transferred to soil in the greenhouse. Plants are tested for expression of the PMI gene using a modified 48-well chlorophenol red assay where the media contains no sucrose and 0.5% mannose. Leaf samples (˜5 mm×5 mm) are placed on this assay media and grown in the dark for ˜72 hours. If the plant is expressing the PMI gene, it can metabolize the mannose and the media will turn yellow. If not, the media will remain red. Transformation events have also been created using Type I callus obtained from immature zygotic embryos using standard culture techniques. For gene delivery, approximately 300 mg of the Type I callus is prepared by subculturing to fresh media 1 to 2 days prior to gene delivery, selecting target tissue pieces and placing them in a ring pattern 10 mm from the center of the target plate on medium again containing 12% sucrose. After approximately 4 hours, the tissue is bombarded using the PDS-1000/He Biolistic device from DuPont. The plasmids to be transformed are precipitated onto 1 μm gold particles using the standard protocol from DuPont. Genes are delivered using two shots per target plate at 650 psi. Approximately 16 hours after gene delivery the callus is transferred to standard culture medium containing 2% sucrose with no selection agent. At 12 or 13 days after gene delivery, target tissue pieces are transferred to selection media containing 40 mg/l phosphinothricin as either Basta or bialaphos. The callus is subcultured on selection for 12 to 16 weeks, after which surviving and growing callus is transferred to standard regeneration medium containing 3 mg/l phosphinothricin as Basta for the production of plants.

### Example 14: Transformation of Soybean

Protoplasts of Glycine max are prepared by the methods as described by Tricoli et al., 1986 (Plant Cell Rep. 5: 334-337), or Chowhury and Widholm, 1985 (Plant Cell Rep. 4: 289-292), or Klein et al., 1981 (Planta 152: 105-114). The protoplast suspension is distributed as 1 ml aliquots into plastic disposable cuvettes. For transformation, 10 μg of DNA is added in 10 μl sterile distilled water and sterilized as described by Paszkowski et al., 1984 (EMBO J. 3:2717-2722). The solution is mixed gently and then subjected at room temperature (24 to 28° C.) to a pulse of 400 Vcm⁻¹ with an exponential decay constant of 10 ms from a BTX-Transfector 300 electroporation apparatus using the 471 electrode assembly.

The above is repeated with one or more of the following modifications:

(1) The voltage used is 200 Vcm⁻¹ or between 100 Vcm⁻¹ and 800 Vcm⁻¹

(2) The exponential decay constant is 5 ms, 15 ms or 20 ms

(3) 50 μg of sheared calf thymus DNA in 25 μl sterile water is added together with the plasmid DNA

(4) The plasmid DNA is linearized before use by treatment with an appropriate restriction enzyme (e.g. BamHI)

The protoplasts are cultured as described in Klein et al., 1981 (Planta 152: 105-114), Chowhury and Widholm, 1985 (Plant Cell Rep. 4: 289-292), or Tricoli et al., 1986 (Plant Cell Rep. 5: 334-337), without the addition of alginate to solidify the medium.

### Example 15: Transformation of Cotton

Agrobacterium strains containing the binary vectors for transformation that are constructed by standard methods are grown 18 to 24 hours in glutamate salts media adjusted to pH 5.6 and supplemented with 0.15% mannitol, 50 μg/ml kanamycin, 50 μg/ml spectinomycin and 1 mg/ml streptomycin before they are diluted to an OD₆₀₀ of 0.2 in the same media without the antibiotics. The bacteria are then grown for three to five hours before dilution to an OD600 of 0.2 to 0.4 and then used for inoculation of discs cut from surface sterilized cotton seeds. The cotton seeds are soaked 20 min in 10% chlorox and rinsed with sterile water. The seeds are germinated on 0.7% water agar in the dark. The seedlings are grown for one week before inoculation of the bacteria onto the cotyledon surface. The inoculated cotyledons are allowed to form callus before they are cut and placed on 0.7% agar containing MS salts, 3% sucrose, 100 μg/ml carbenicilfin, and 100 μg/ml mefoxim. The callus is transferred to fresh media every three weeks until sufficient quantity is obtained for 4 plates. Half of the callus growing from the virulent Agrobacterium strains is transferred to media without hormones containing 50 μg/ml kanamycin.

### Example 16: Aspergillus flavus/Aflatoxin Disease Assay

Inoculum Production and Inoculation Protocol:

Inoculum is an equal mixture of conidia from four highly virulent isolates of Aspergillus flavus. Each isolate is grown separately in petri dishes on potato-dextrose agar for 12 to 16 days at 28° C. with 12 h light. Cultures, including media, are blended with distilled water and filtered through double layered cheese cloth. Conidial concentrations are estimated using a hemacytometer and adjusted with distilled water. Two drops of Tween 20 per 100 ml are added as a surfactant. Conidial suspensions are prepared immediately prior to use and stored on ice while transporting from the lab to the field. Primary ears of each plant are inoculated 20-24 days at the midsilk growth stage (50 percent of the ears with emerged silks) with a spore suspension of 1×10⁶ conidia/ml using a pin-board inoculator [Plant Disease (1994) 78:778-781].

Ear Rot Rating and Aflatoxin Analysis:

Forty to forty-five days after inoculation, ears are husked and a visual disease severity rating of 1 to 9 (1=no disease, 9=90-100 percent infected) is made for each inoculated ear and averaged for each plot. If necessary, ears are dried by forced air and shelled. Kernels for each plot are bulked and subsampled for mycotoxin analysis. The subsamples are ground with a Romer Mill (Model 2A) and analyzed for total aflatoxin by thin layer chromatography using standard AOAC methods.

### Example 17: Construction of Plant Transformation Vectors Containing 5′-Promoter Fragments Operably Linked to GUS or GFP Reporter Genes

To produce promoter::reporter fusions, pBSK+LOX4A (see Example 9) is used as template for the polymerase chain reaction (PCR). Gene-specific primers are used to amplify the 5′-promoter region of the gene. Using combinations of the reverse primer R1 (SEQ ID NO:12) with forward primers F1 (SEQ ID NO:13) and F2 (SEQ ID NO:14) the regulatory sequences that are ˜1.2 kb and ˜2 kb upstream of the initiating methionine are isolated. The nucleotide sequence of the PCR fragment amplified with forward primer F1 and reverse primer R1 is shown in SEQ ID NO:18, and the nucleotide sequence of the PCR fragment amplified with forward primer F2 and reverse primer R1 is shown in SEQ ID NO:19. For ease of cloning the primers consist of gene specific sequences and attb recombination sites for the GATEWAY™ cloning technology (Life Technologies, Invitrogen Corporation, Carlsbad, Calif. USA). As reverse primer, primer R1 is used, which has the following sequence: 5′-CAAGAAAGCTGGGTTGACAAATTAAGTTGTCAGTGTG-3′ (SEQ ID NO:12). The gene specific sequence of reverse primer R1 is underlined (corresponds to position 1356 to 1334 of SEQ ID NO:2), the attB recombination sequence is denoted in italics. Forward primers are the primers F1 and F2. Forward primer F1 has the following sequence: 5′-CAAAAAAGCAGGCTTGTAACATCCTACTCCTATTGTG-3′ (SEQ ID NO:13). The gene specific sequence of forward primer F1 is underlined (corresponds to bases 159 to 181 of SEQ ID NO:2), the attB recombination sequence is denoted in italics. F1 in combination with R1 amplifies a fragment of ˜1.2 kb. Forward primer F2 has the following sequence: 5′-CAAAAAAGCAGGCTCCCCGTCTTTATCTACTC-3′ (SEQ ID NO:14). The gene specific sequence of forward primer F2 is underlined (corresponds to bases 31 to 48 of SEQ ID NO: 1), the attB recombination sequence is denoted in italics. Primer F2 in combination with primer R1 amplifies a fragment of ˜2 kb. Using a nested PCR strategy the regulatory sequence is amplified first with primers F1+R1 or F2+R1 followed by a second PCR with primer attB1 (5′-GGGGACAAGTTTGTACAAAAAAGCAGGCT-3′, SEQ ID NO:15) and primer attB2 (5′-GGGGACCACTTTGTACAAGAAAGCTGGGT-3′, SEQ ID NO:16). The following PCR conditions are used with gene-specific primers F1+R1 or F2+R1: ((94° C.:15min):(94° C.:10 sec/53° C.:10 sec/72° C.:1 min)×15:(72° C.:2 min)). Following PCR the products are used in a second PCR reaction for amplification with the attBl+attB2 primers. In the subsequent PCR amplification, the following PCR conditions are used: ((94° C.:15sec):(94° C.:15sec/68° C.:2 min,15 sec)×25:( 68° C.:3 min). The resulting PCR product are then flanked by attB recombination sites and are used to generate Entry Clones in pDONR201 via the BP reaction according to manufacturers protocol (see: Instruction Manual of GATEWA™ Cloning Technology, GIBCO BRL, Rockville, Md. USA, http://www.lifetech.com/). The resulting plasmids contain ˜1.2 kb and ˜2 kb 5′ of the RCI-1 initiation codon and are referred to as pENTR+LOXp1.2, pENTR+LOXp2.

1. Produced Entry Vector Constructs

pENTR+LOX1.2 (pDONR201+1.2 kb promoter fragment flanked by att recombination sequences)

pENTR+LOX2 (pDONR201+2 kb promoter fragment flanked by att recombination sequences)

These entry vectors are used to construct a binary promoter::reporter plasmid for maize or rice transformation. The regulatory/promoter sequence is fused to the GUS reporter gene (Jefferson et al, 1987, EMBO J 6: 3901-3907) or to GFP by recombination using GATEWAY™ Technology according to manufacturers protocol as described in the Instruction Manual (GATEWAY™ Cloning Technology, GIBCO BRL, Rockville, Md. http://www.lifetech.com/). Briefly, according to this protocol the promoter fragment in the entry vector is recombined via the LR reaction with a binary Agrobacterium destination vector containing the GUS coding region with intron or GFP that have an attR site 5′ to the GUS or GFP reporter gene (pNOV2347 or pNOV2361, respectively). The orientation of the inserted fragment is maintained by the att sequences and the final construct is verified by sequencing. The construct is then transformed into Agrobacterium tumefaciens strains by electroporation.

pNOV2347 and pNOV2361 are binary vectors with VS1 origin of replication, a copy of the Agrobacterium virG gene in the backbone and a Maize Ubiquitin promoter- PMI gene-nos terminator expression cassette between the left and right borders of T-DNA. PMI (phosphomannose isomerase) is the coding region of the E. coli manA gene (Joersbo and Okkels, 1996, Plant Cell Reports 16:219-221, Negrotto et al., 2000, Plant Cell Reports 19:798-803). The nos (nopaline synthase) terminator is obtained from Agrobacterium tumefaciens T-DNA (Depicker et al., 1982, J. Mol. Appl. Genet.1 (6), 561-573). The maize ubiquitin promoter, the phosphomannose isomerase coding region and the nos terminator are located at nt 4114 to nt 5114, nt 6192 to nt 7295 and nt 7356 to 7604 respectively, of pNOV2347 (SEQ ID NO: 20). pNOV2361 is identical to pNOV2347, except that pNOV2361 has a GFP instead of a GUS reporter gene. The reporter-promoter cassettes are inserted closest to the right border. The selectable marker expression cassette in the binary vectors is closest to the left border. The vector contains GATEWAY™ recombination components which were introduced into the binary vector backbone by ligating a blunt-ended cassette containing attR sites, ccdB and chloramphenicol resistance marker using the GATEWAY™ Vector Conversion System (LifeTechnologies, www.lifetech.com.). The GATEWAY™ cassette is located between nt 2351 and 4050 (complementary) of pNOV2347 and between nt 9201 and 10910 of pNOV2361. The promoter cassettes are inserted through an LR recombination reaction (LifeTechnologies, www.lifetech.com.) whereby the DNA sequence of pNOV2347 between nt 2351 and nt 4050 is removed and replaced with the LOX promoter fragment flanked by att sequences. The recombination results in the promoter sequence fused to the GFP or GUS reporter gene with intron (GIG) sequence. The GIG gene contains the ST-LS1 intron from Solanum tuberosum at nt 385 to nt 576 of GUS (SEQ ID NO:21) (obtained from Dr. Stanton Gelvin, Purdue University, and described in Narasimhulu, et al 1996, Plant Cell, 8: 873-886.). Shown below are the orientations of the selectable marker and promoter-reporter cassettes in the binary vector constructs.

2. Produced Constructs for Stable Transformation

pNOV6800 (RB nos+GlG gene+LOX1.2 promoter fragment-ZmUbi+PMI gene+nos LB)

pNOV6801 (RB LOX1.2 promoter fragment+GFP gene+nos-ZmUbi+PMI gene+nos LB)

The nucleotide sequence of pNOV6800 is depicted in SEQ ID NO:22. pNOV6800 and pNOV6801 differ only in the expression cassette located between the right and left borders of the binary vector.

3.Constructs Used for Comparison

pNOV2110 (RB ZmUbi Promoter+GFP gene+nos-ZmUbi+PMI gene+nos LB)

pNOV 3640(RB nos-GIG-ZmUbi promoter nos-AtPPOdm-ZmUbi promoter LB) GUS—Intron—GUS, GFP and polyA fragments are identical to those used for the LOX promoter constructs above. The ZmUbi promoter corresponds to the fragment from base 12 to base 2009 in pNOV2110 and contains promoter, Exon1 and Intron1 of the Maize Ubiquitin gene. The AtPPOdm sequence encodes a mutated form of the protophorinogen oxidase protein which confers resistance to herbicides (PPO inhibitors) that normally inactivate the enzyme (U.S. Pat. No. 5,939,602).

### Example 18: Agrobacterium-Mediated Transformation of Maize

Transformation of immature maize embryos is performed essentially as described in Negrofto et al., (2000) Plant Cell Reports 19: 798-803. For this example, all media constituents are as described in Negrotto et al., supra. However, various media constituents described in the literature may be substituted.

1. Transformation Plasmids and Selectable Marker

The genes used for transformation are cloned into a vector suitable for maize transformation as described in Example 17. Vectors used contain the phosphomannose isomerase (PMI) gene (Negrotto et al. (2000) Plant Cell Reports 19: 798-803) as a selectable marker.

2. Preparation of Agrobacterium tumefaciens

Agrobacterium strain LBA4404 (pSB1) containing the plant transformation plasmid is grown on YEP (yeast extract (5 g/L), peptone (10 g/L), NaCl (5 g/L),15 g/l agar, pH 6.8) solid medium for 2 to 4 days at 28° C. Approximately 0.8×10⁹ Agrobacteria are suspended in LS-inf media supplemented with 100 μM acetosyringone (As) (LSAs medium) (Negrotto et al., (2000) Plant Cell Rep 19: 798-803). Bacteria are pre-induced in this medium for 30-60 minutes.

3. Inoculation

Immature embryos from A188 or other suitable maize genotypes are excised from 8-12 day old ears into liquid LS-inf+100 μM As (LSAs). Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) (Negrotto et al. 2000) and cultured in the dark for 28° C. for 10 days.

4. Selection of Transformed Cells and Regeneration of Transformed Plants

Immature embryos producing embryogenic callus are transferred to LSD1M0.5S medium (LSDc with 0.5 mg/l 2,4-D instead of Dicamba, 10 g/l mannose, 5 g/l sucrose and no silver nitrate). The cultures are selected on this medium for 6 weeks with a subculture step at 3 weeks. Surviving calli are transferred either to LSD1MO.5S medium to be bulked-up or to Reg1 medium (as described in Negrotto et al., 2000). Following culturing in the light (16 hour light/8 hour dark regiment), green tissues are then transferred to Reg2 medium without growth regulators (as described in Negrotto et al. 2000) and incubated for 1-2 weeks. Plantlets are transferred to Magenta GA-7 boxes (Magenta Corp, Chicago Ill.) containing Reg3 medium (as described in Negrotto et al. 2000) and grown in the light. Plants that are PCR positive for the promoter-reporter cassette are transferred to soil and grown in the greenhouse.

### Example 19: GUS Reporter Gene Assays

Promoter activity is evaluated qualitatively and quantitatively using histochemical and florescence assays for expression of the B-glucuronidase (GUS) enzyme.

1. Histochemical β-glucuronidase (GUS) Assay

For qualitative evaluation of promoter activity, various tissues and organs are used in GUS histochemical assays. Either whole organs or pieces of tissue are dipped into GUS staining solution. GUS staining solution contains 1 mM 5-bromo-4-chloro-3-indolyl glucuronide (X-Gluc, Duchefa, 20 mM stock in DMSO), 100 mM Na-phosphate buffer pH 7.0, 10 mM EDTA pH 8.0, and 0.1% Triton X100. Tissue samples are incubated at 37° C. for 1-16 hours. If necessary samples can be cleared with several washes of 70% EtOH to remove chlorophyll. Following staining tissues are viewed under a light microscope to evaluate the blue staining showing the GUS expression pattern.

2. β-Glucuronidase (GUS) Fluorescence Assay

For quantitative analysis of promoter activity in various tissues and organs, GUS expression is measured fluorometrically. Tissue samples are harvested and ground in ice cold GUS extraction buffer (50 mM Na₂HPO₄ pH 7.0, 5mM DTT, 1mM Na₂EDTA, 0.1% TritonX100, 0.1% sarcosyl). Ground samples are spun in a microfuge at 10,000 rpm for 15 minutes at 4° C. Following centrifugation the supernatant is removed for GUS assay and for protein concentration determination. To measure GUS activity the plant extract is assayed in GUS assay buffer (50 mM Na₂HPO₄ pH 7.0, 5 mM DTT, 1 mM Na₂EDTA, 0.1% TritonX100, 0.1% sarcosyl, 1 mM 4-Methylumbelliferyl-beta-D-glucuronic acid dihydrate (MUG)), prewarmed to 37° C. Reactions are incubated and 100 μl aliquots are removed at 10 minute intervals for 30 minutes to stop the reaction by adding to tubes containing 900 μL of 2% Na₂CO₃. The stopped reactions are then read on a Tecan Spectroflourometer at 365 nm excitation and 455 nm emission wavelengths. Protein concentrations are determined using the BCA assay following manufacturers protocol. GUS activity is expressed as relative florometric units (RFU)/mg protein.

### Example 20: GFP Reporter Gene Assays

Promoter activity is evaluated qualitatively using microscopic imaging fluorescence and quantitatively using fluorescence assays for expression of the green florescent protein.

1. Microscopic Evaluation of GFP Expression

Expression of the promoter::GFP fusion is monitored in transformants by microscopic imaging using a Leica MzFLIII fluorescence microscope (Leica Microsystems, Heidelberg, Germany) with GFP2 and GFP3 filter settings.

2. Quantitative GFP Fluorometric Assay

To assay expression of GFP in tissues of transgenic plants, harvested tissue is frozen and frozen tissue is ground thoroughly. Following grinding add 300 μL of extraction buffer (EB; 10 mM Tris-HCL (pH7.5), 100 mM NaCl, 1 mM MgCI₂, 10 mM DTT and 0.1% Sarcosyl). Vortex well to mix sample, centrifuge for 10 minutes, then transfer the supernatant to a new tube or microtitre plate well for reading. The sample tube or plate is then inserted into the Tecan Spectroflourometer plate reader set to 465 nm excitation and 512 nm emissions wavelengths for 10 flashes and a gain of 100. If the expression levels are too high resulting in some of the samples being over the limit (says VALUE in the cell) then parameters are changed to 8 flashes and a gain of 80. Protein concentrations are determined using the BCA assay following manufacturers protocol. GFP activity is expressed as the relative fluorescence units (RFU) per milligram of protein.

### Example 21: Evaluation of Promoter Activity

To evaluate activity of the promoter::reporter fusion, tissue samples are harvested from untreated control plants as well as from plants treated with the chemical activator, BTH or jasmonic acid. In transgenic rice treatment with the chemical inducers is done 10 days after sowing Ti seed at the emergence of leaf 3. In transgenic maize treatment with the chemical inducers is done three weeks after sowing Ti seed. All chemical concentrations are given as ppm (mg active ingredient I⁻¹ of applied solution). Probenazole is applied as a 250 ppm solution of the pure substance by soil drench as described (Thieron et al. (1995) Systemic acquired resistance in rice: Studies on the mode of action of diverse substances inducing resistance in rice to Pyricularia oryzae. Mededelingen Faculteit Landbouwkundige en Toegepaste Biologische Wetenschappen Universiteit Gent. 60, 421-430). Formulations of BTH (1:1 (w/w) mixture of active ingredient and wettable powder) is applied onto leaves by spraying. All controls are done by application of spray-solutions without active substance. Jasmonic acid is applied as a 1 mM solution in ethanol as described (Schweizer et al. (1997) Plant Physiol. 114, 79-88). Wounding and measurement of gene expression in systemic tissue is done according to (Schweizer et al. (1998) Plant J. 14, 475-481).

Various modifications of the invention in addition to those shown and described herein will become apparent to those skilled in the art from the foregoing description and examples. Such modifications are intended to fall within the scope of the appended claims.

Various references and patents have been cited herein, and are all incorporated by reference in their entireties.

What is claimed is:

1. An isolated nucleic acid molecule capable of driving chemically-inducible but not wound- or pathogen-inducible expression of an associated nucleotide sequence.

2. The isolated nucleic acid molecule according to claim 1, wherein said nucleic acid molecule is a component of the Pstl/Pstl fragment of about 4.5 kb in length from plasmid pBSK+LOX4A which has been deposited under accession no DSM 13524.

3. The isolated nucleic acid molecule according to claim 1, wherein said nucleic acid molecule is a component of the nucleotide sequence depicted in SEQ ID NO:17.

4. The isolated nucleic acid molecule according to claim 3, wherein said nucleic acid molecule is depicted in SEQ ID NO:18.

5. The isolated nucleic acid molecule according to claim 3, wherein said nucleic acid molecule is depicted in SEQ ID NO:19.

6. The isolated nucleic acid molecule according to claim 1, wherein said nucleic acid molecule comprises the nucleotide sequence depicted in SEQ ID NO:1.

7. The isolated nucleic acid molecule according to claim 1, wherein said nucleic acid molecule comprises nt 1 to nt 1358 of the nucleotide sequence depicted in SEQ ID NO:2.

8. The isolated nucleic acid molecule according to claim 1, wherein said nucleic acid molecule comprises nt 1702 to nt 2104 of SEQ ID NO:2 and/or nt 1 to nt 97 of SEQ ID NO:3 and/or nt 367 to nt 1283 of SEQ ID NO:3 of SEQ ID NO:3.

9. The isolated nucleic acid molecule according to claim 1 or 2, wherein said nucleic acid molecule comprises a combination of any one of the nucleotide sequences or portions thereof depicted in SEQ ID NO:1, SEQ ID NO:2 and SEQ ID NO:3.

10. An isolated nucleic acid molecule which hybridizes under stringent conditions to SEQ ID NO:1, SEQ ID NO:2, SEQ ID NO:3, SEQ ID NO:17, SEQ ID NO:18 or SEQ ID NO:19, or to the 4.5 kb Pstl fragment of plasmid pBSK+LOX4A which has been deposited under accession no DSM 13524, wherein said isolated nucleic acid molecule is capable of driving chemically-inducible but not wound- or pathogen-inducible expression of an operably-linked nucleotide sequence.

11. An isolated nucleic acid molecule comprising a consecutive stretch of at least 50 nt of SEQ ID NO:1, SEQ ID NO:2, SEQ ID NO:3, SEQ ID NO:17, SEQ ID NO:18 or SEQ ID NO:19, or of the 4.5 kb Pstl fragment of plasmid pBSK+LOX4A which has been deposited under accession no DSM 13524, wherein said isolated nucleic acid molecule is capable of driving chemically inducible but not wound- or pathogen-inducible expression of an operably-linked nucleotide sequence.

12. An isolated nucleic acid molecule according to claim 11 wherein said consecutive stretch of at least 50 nt has at least 70% sequence identity with a consecutive stretch of at least 50 nt of SEQ ID NO:1, SEQ ID NO:2, SEQ ID NO:3, SEQ ID NO:17, SEQ ID NO:18 or SEQ ID NO:19, or of the 4.5 kb Pstl fragment of plasmid pBSK+LOX4A which has been deposited under accession no DSM 13524.

13. The isolated nucleic acid molecule according to any one of claims 1 to 12, wherein the chemical inducer is selected from the group consisting of BTH (benzo(1,2,3)thiadiazole-7-carbothioic acid S-methyl ester), INA (2,6-dichloroisonicotinic acid) and probenazole.

14. The isolated nucleic acid molecule according to any one of claims 1 to 12, wherein the chemical inducer is jasmonic acid.

15. A recombinant nucleic acid molecule comprising the isolated nucleic acid molecule according to any one of claims 1 to 14 operably linked to a nucleotide sequence of interest.

16. The recombinant nucleic acid molecule of claim 15, wherein the nucleotide sequence of interest comprises a polypeptide coding sequence.

17. The recombinant nucleic acid molecule of claim 16, wherein the coding sequence comprises at its 5′-end a nucleotide sequence encoding the amino acid sequence depicted in SEQ ID NO:6.

18. The recombinant nucleic acid molecule of claim 16 or 17, wherein the coding sequence encodes a desirable phenotypic trait.

19. The recombinant nucleic acid molecule of claim 16, wherein the coding sequence is in antisense orientation.

20. A nucleic acid expression vector comprising the isolated nucleic acid molecule according to any one of claims 1 to 14 or the recombinant nucleic acid molecule of any one of claims 15 to 19.

21. A host cell stably transformed with an isolated nucleic acid molecule according to any one of claims 1 to 14 or a recombinant nucleic acid molecule of any one of claims 15 to 19.

22. The host cell of claim 21, wherein said host cell is a plant cell.

23. A plant and the progeny thereof stably transformed with an isolated nucleic acid molecule according to any one of claims 1 to 14 or a recombinant nucleic acid molecule of claims 15 to 19.

24. The plant of claim 23, wherein said plant is selected from the group consisting of maize, wheat, sorghum, rye, oats, turf grass, rice, barley, soybean, cotton, tobacco, sugar beet and oilseed rape.

25. Use of the nucleic acid molecule of any one of claims 1 to 14 to express a nucleotide sequence of interest.

26. A method of producing the isolated nucleic acid molecule according to claim 1, wherein the isolated nucleic acid molecule is produced by a polymerase chain reaction wherein at least one oligonucleotide used comprises a sequence of nucleotides which represents a consecutive stretch of 15 or more nucleotides of SEQ ID NO:1, SEQ ID NO:2 SEQ ID NO:3, SEQ ID NO:17, SEQ ID NO:18 or SEQ ID NO:19.

27. An isolated nucleic acid molecule encoding the amino acid sequence depicted in SEQ ID NO:6, wherein said amino acid sequence is capable of targeting an associated protein to plastids.

28. The isolated nucleic acid molecule according to claim 27, wherein said isolated nucleic acid molecule is depicted in SEQ ID NO:4.

29. An isolated nucleic acid molecule which hybridizes under stringent conditions to the nucleotide sequence depicted in SEQ ID NO:4.

30. A peptide encoded by the nucleotide sequence of any one of claims 27 to 29.

31. A peptide encoded by the nucleotide sequence of claim 28.

32. Use of the peptide of claim 30 or 31 to target an associated protein of interest to plastids.

33. An isolated nucleic acid molecule which hybridizes under stringent conditions to SEQ ID NO:5, wherein the protein encoded by said DNA has at least 65% amino acid sequence identity with the amino acid sequence depicted in SEQ ID NO:7 and encodes a protein with lipoxygenase activity.

34. The isolated nucleic acid molecule according to claim 33, wherein said nucleotide sequence encodes the protein depicted in SEQ ID NO:7.

35. A protein encoded by the nucleotide sequence of the isolated nucleic acid molecule of claim 33 or 34.

36. A protein encoded by the nucleotide sequence of the isolated nucleic acid molecule of claim 34.

37. Use of the protein according to claim 35 or 36 to inhibit fungal mycotoxins.

38. A method of inhibiting fungal mycotoxins in a plant by transforming the plant with nucleic acid molecule of claim 33 or 34 and expressing a polypeptide having lipoxygenase activity.

39. A recombinant nucleic acid molecule comprising the nucleic acid molecule according to any one of claims 27 to 29 or claims 33 to 34.

40. A host cell stably transformed with the recombinant nucleic acid molecule of claim 39.

41. The host cell of claim 40, wherein said host cell is a plant cell.

42. A plant and the progeny thereof stably transformed with the recombinant nucleic acid molecule of claim 39.

43. The plant of claim 42, wherein said plant is selected from the group consisting of maize, wheat, sorghum, rye, oats, turf grass, rice, barley, soybean, cotton, tobacco, sugar beet and oilseed rape.

44. A seed of the plant and progeny thereof of claim 43.
